(19) United States
(12) Patent Application Publication
(10) Pub. No.: US 2018/0140475 A1
(43) Pub. Date: May 24, 2018

(54) ABSORBENT ARTICLE WITH NON-INTEGRAL SIDE COVERS AND NON-INTEGRAL WINGS

(71) Applicant: Kimberly-Clark Worldwide, Inc., Neenah, WI (US)

(72) Inventors: JuHyung Lee, Gangnam-gu, (KR); SungSu A. Kim, Gangnam-gu (KR); MoonYoung Cho, Yongin-si, Gyeonggi-do (KR); SeoYeon Son, Gangnam-gu (KR); HyungWoo Park, Gangnam-gu (KR)

(21) Appl. No.: 15/575,154

(22) PCT Filed: May 29, 2015
(86) PCT No.: PCT/US15/33194

§ 371 Date: Nov. 17, 2017

(51) Int. Cl. A61F 13/47; A61F 13/472; A61F 13/56

(52) U.S. Cl. CPC A61F 13/4704; A61F 13/47236; A61F 13/47272; A61F 13/515; A61F 13/5611; A61F 13/47218; A61F 13/5616

(57) ABSTRACT

An absorbent article can have a chassis which can have a backsheet layer, a topsheet layer, and an absorbent system positioned between the backsheet layer and the topsheet layer. A portion of the backsheet layer can be bonded to a portion of the topsheet layer in an area. A pair of non-integral side covers can be bonded to the chassis in a position which overlays the bond between the backsheet layer and the topsheet layer. A pair of non-integral wings can be bonded to the backsheet layer of the chassis and are separate from the side covers.

### BACKGROUND

Products such as absorbent articles are often used to collect and retain human body exudates containing, for example, urine, menses and/or blood. Comfort, absorbency, and discretion are three main areas of concern for the wearer of the product. In particular, a wearer is often interested in knowing that such products will absorb significant volumes of body exudates with minimal leakage in order to protect their undergarments, outer garments, or bedsheets from staining, and that such products will help them avoid the subsequent embarrassment brought on by such staining.

Currently, a wide variety of products for absorption of body exudates are available in the form of feminine pads, sanitary napkins, panty shields, pantiliners, and incontinence devices. These products generally have an absorbent core positioned between a wearer-facing liquid permeable topsheet layer and a garment-facing liquid impermeable backsheet layer. The edges of the topsheet and the backsheet layers are often bonded together at their periphery to form a seal to contain the absorbent core and body exudates received into the product through the topsheet layer. In use, such products are typically positioned in the crotch portion of an undergarment for absorption of the body exudates and a garment attachment adhesive on the backsheet layer can be used to attach the product to the inner crotch portion of the undergarment. Some of these products can also include wings for wrapping about the wearer's undergarment to further secure the product to the undergarment and to protect the undergarment from staining. Such wings (also known as flaps or tabs) are frequently made from lateral extensions of the topsheet and/or backsheet layers.

Such products, however, can have drawbacks which can detract from the comfort, absorbency and discretion sought by the wearer of the product. For example, such products generally have a crotch region which will have a defined crotch width—a maximum crotch width to allow for wearer comfort during usage and a minimum crotch width for leakage protection during usage. The defined crotch width of the product, however, may not correspond to the crotch width of the undergarment chosen for wear by the wearer of the product. As a result, the product may extend, in the width direction, beyond the edges of the undergarment resulting in discomfort to the wearer or the product may be narrower, in the width direction, than the crotch width of the undergarment which can result in leakage and potential staining of the undergarment.

A product having wings and a crotch width which does not correspond to the crotch width of the chosen undergarment can also result in difficulty in attaching the product to the undergarment of the wearer. As described above, such wings are frequently made from lateral extensions of the topsheet and/or backsheet layers and are, therefore, integral with the product. Products with wings are generally provided to a wearer in a folded pre-use configuration wherein the wings are folded over either of the topsheet layer or the backsheet layer. The folded pre-use configuration, therefore, incorporates fold regions into the product between the wings and the main body portion of the product. As the wearer of the product places the product into their undergarment, the wearer generally uses the fold regions between the wings and the main body portion as a positioning guide as the wearer will generally position the side seams of the crotch portion of their undergarment into the fold regions between the wings and the main body portion of the product. The width between the fold regions, however, may not correspond to the crotch width of the undergarment chosen by the wearer of the product. As the wearer attempts to place the product in their undergarment, pulling of the wings to wrap around the undergarment in an attempt to correspond the width between the fold regions to the width of their undergarment can result in a distortion, bunching, twisting, and/or poor attachment of the product which can be uncomfortable to the wearer and may also result in leakage of body exudates from the absorbent article. An additional drawback is that the peripheral seal between the topsheet layer and the backsheet layer can irritate the skin of the wearer which can be exacerbated by any additional distortion, bunching, twisting, and/or poor attachment of the product.

There remains a need for an improved product, such as an absorbent article, that can better correspond to various crotch widths of undergarments. There remains a need for an improved product, such as an absorbent article, that can have minimal distortion, bunching and/or twisting in the undergarment of the wearer. There remains a need for an improved product, such as an absorbent article, that can cause minimal irritation to the wearer's skin during usage of the absorbent article.

### SUMMARY

In various embodiments, an absorbent article can have a longitudinal direction, a transverse direction, and a depth direction; a longitudinal axis and a transverse axis wherein the absorbent article is asymmetrical about the transverse axis; a chassis comprising: a first end edge and a second end edge; a topsheet layer comprising a first longitudinal direction peripheral edge and a second longitudinal direction peripheral edge; a backsheet layer comprising a first longitudinal direction peripheral edge and a second longitudinal direction peripheral edge; an absorbent system positioned between the topsheet layer and the backsheet layer, the absorbent system comprising an absorbent core, a garment facing surface, a wearer facing surface, and a first longitudinal direction peripheral region and a second longitudinal direction peripheral region; a first longitudinal direction bond region wherein the first longitudinal direction peripheral edge of the topsheet layer is bonded to the first longitudinal direction peripheral edge of the backsheet layer; and a second longitudinal direction bond region wherein the second longitudinal direction peripheral edge of the topsheet layer is bonded to the second longitudinal direction peripheral edge of the backsheet layer; a first side cover bonded to the chassis in the region of the first longitudinal direction bond region and a second side cover bonded to the chassis in the region of the second longitudinal direction bond region; and a first non-integral wing and a second non-integral wing, each of the first and second non-integral wings bonded to the backsheet layer.

In various embodiments, the first non-integral wing and the second non-integral wing are separate components from each other. In various embodiments, the first non-integral wing and the second non-integral wing are integral with a bridge which is bonded to the backsheet layer.

In various embodiments, the first side cover and the second side cover are separate components from each other. In various embodiments, the first side cover and the second side cover are integral with a bridge which is bonded to the topsheet layer.

In various embodiments, the first longitudinal direction bond region is located in the vicinity of the first longitudinal direction peripheral region and the second longitudinal direction bond region is located in the vicinity of the second longitudinal direction peripheral region. In various embodiments, the first longitudinal direction bond region and the second longitudinal direction bond region each overlay a portion of the wearer facing surface of the absorbent system.

In various embodiments, each of the first side cover and the second side cover is bonded to an exterior surface of the backsheet layer. In various embodiments, each of the first side cover and the second side cover is further bonded to a portion of the topsheet layer. In various embodiments, each of the first side cover and the second side cover extend between the first end edge and the second end edge of the chassis.

In various embodiments, each of the first side cover and the second side cover have at least one concave region and at least convex region. In various embodiments, the first end edge has at least one convex region and the second end edge has at least one concave region. In various embodiments, the first end edge and the second end edge form a nesting configuration.

In various embodiments, the chassis further comprises at least one flexure feature. In various embodiments, the at least one flexure feature comprises at least one flexure element extending in a direction generally parallel to the longitudinal axis. In various embodiments, the at least one flexure feature comprises a second flexure element, the second flexure element spaced in the transverse direction outward from the first flexure element and positioned at an angle relative to the longitudinal axis. In various embodiments, the absorbent article further comprising a garment attachment on an exterior surface of the backsheet layer wherein the backsheet layer is substantially free of the garment attachment in an area of the backsheet layer positioned below the flexure feature in the depth direction of the absorbent article.

In various embodiments, the absorbent core is a single layer of material. In various embodiments, the absorbent core is formed of two layers of material. In various embodiments, the absorbent system further comprises at least one additional layer selected from a surge layer, a fluid intake layer, a transfer delay layer and a distribution layer.

### BRIEF DESCRIPTION OF THE DRAWINGS

FIG. 1 is a top view of an embodiment of an absorbent article.

FIG. 2 is an exploded perspective view of the absorbent article of FIG. 1.

FIG. 3 is a cross-sectional view of the absorbent article of FIG. 1 taken along line 3-3.

FIG. 4 is a top view of an embodiment of an absorbent article.

FIG. 5 is an exploded perspective view of the absorbent article of FIG. 4.

FIG. 6 is a cross-sectional view of the absorbent article of FIG. 4 taken along line 6-6.

FIG. 7 is a top view of an embodiment of an absorbent article.

FIG. 8 is a cross-sectional view of the absorbent article of FIG. 7 taken along line 8-8.

FIG. 9 is a top view of an embodiment of a backsheet layer in an unfolded configuration.

FIG. 10 is a top view of an embodiment of a backsheet layer in a folded configuration.

FIG. 11 is a sectional view of a nesting configuration of a first end edge of an absorbent article with a second end edge of the absorbent article.

FIG. 12 is a perspective view of the absorbent article of FIG. 1 in a tented configuration.

FIG. 13 is a top view of an embodiment of an absorbent article.

FIG. 14 is an exploded perspective view of the absorbent article of FIG. 13.

FIG. 15 is a cross-sectional view of the absorbent article of FIG. 13 taken along line 15-15.

FIG. 16 is a bottom view of an embodiment of an absorbent article.

FIG. 17 is a bottom view of an embodiment of an absorbent article.

### DETAILED DESCRIPTION

The present disclosure is generally directed towards an absorbent article which can have an improved attachment to a wearer's undergarment and which can have a reduction in irritation to a wearer's skin during usage of the absorbent article. The absorbent article can have a chassis, a pair of non-integral side covers, and a pair of non-integral wings. The chassis of the absorbent article can have a topsheet layer, a backsheet layer, and an absorbent system positioned between the topsheet layer and the backsheet layer. The topsheet layer and the backsheet layer can overlay each other and can be bonded together to form a seal between the topsheet layer and the backsheet layer in order to contain the absorbent system and body exudates received into the chassis through the topsheet layer. The non-integral side covers can be bonded to the chassis and can overlay at least a portion of the seal between the topsheet layer and the backsheet layer, such as, for example, a portion of the seal which extends in a longitudinal direction of the absorbent article. The non-integral wings can be bonded to the backsheet layer of the chassis and can extend in a transverse direction of the absorbent article.

### Definitions:

As used herein, the term “absorbent article” refers herein to a garment or other end-use personal care absorbent article, including, but not limited to, catamenial products, such as sanitary napkins, feminine pads, pantiliners, and panty shields, incontinence devices, and the like.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form one fiber. Conjugate fibers are also sometimes referred to as bicomponent fibers or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al. each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10 or 20 gsm to about 120, 125 or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. Nos. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in part on ionicity (a factor of the ionic concentration of the aqueous solution), and the SAP functional polar groups that have an affinity for water. SAP are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating on another material or fiber.

### Absorbent Article:

The present disclosure is generally directed towards an absorbent article which can have an improved attachment to a wearer's undergarment and which can have a reduction in irritation to a wearer's skin during usage of the absorbent article. The absorbent article can have a chassis, a pair of non-integral side covers, and a pair of non-integral wings. The chassis of the absorbent article can have a topsheet layer, a backsheet layer, and an absorbent system positioned between the topsheet layer and the backsheet layer. The topsheet layer and the backsheet layer can overlay each other and can be bonded together to form a seal between the topsheet layer and the backsheet layer in order to contain the absorbent system and body exudates received into the chassis through the topsheet layer. The non-integral side covers can be bonded to the chassis and can overlay at least a portion of the seal between the topsheet layer and the backsheet layer, such as, for example, a portion of the seal which extends in a longitudinal direction of the absorbent article. The non-integral wings can be bonded to the backsheet layer of the chassis and can extend in a transverse direction of the absorbent article.

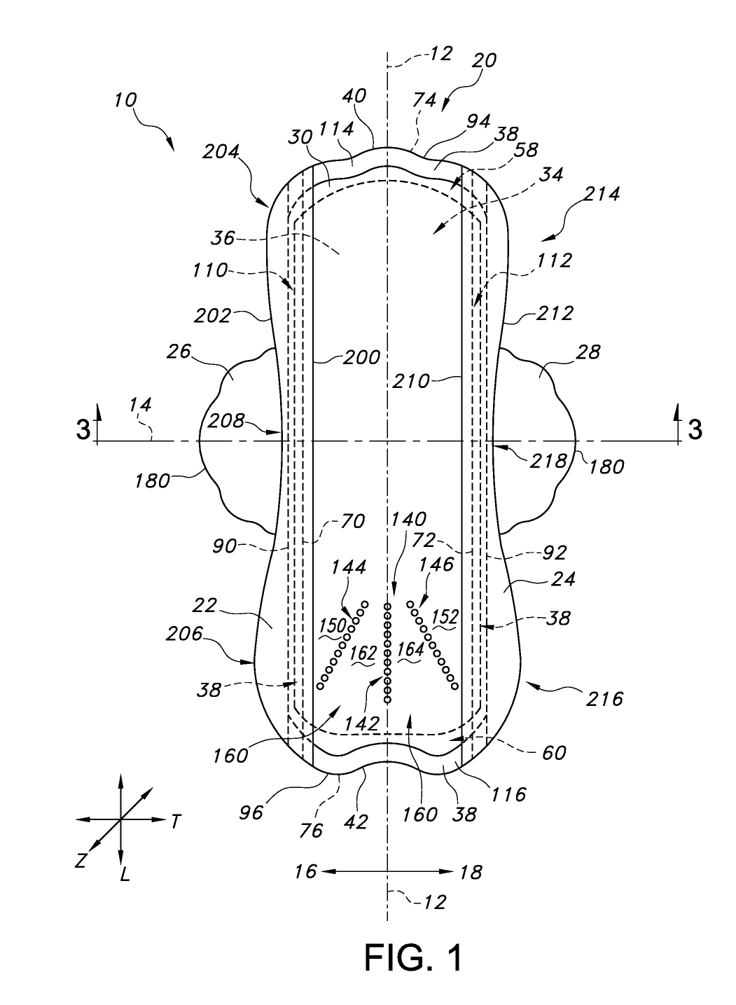

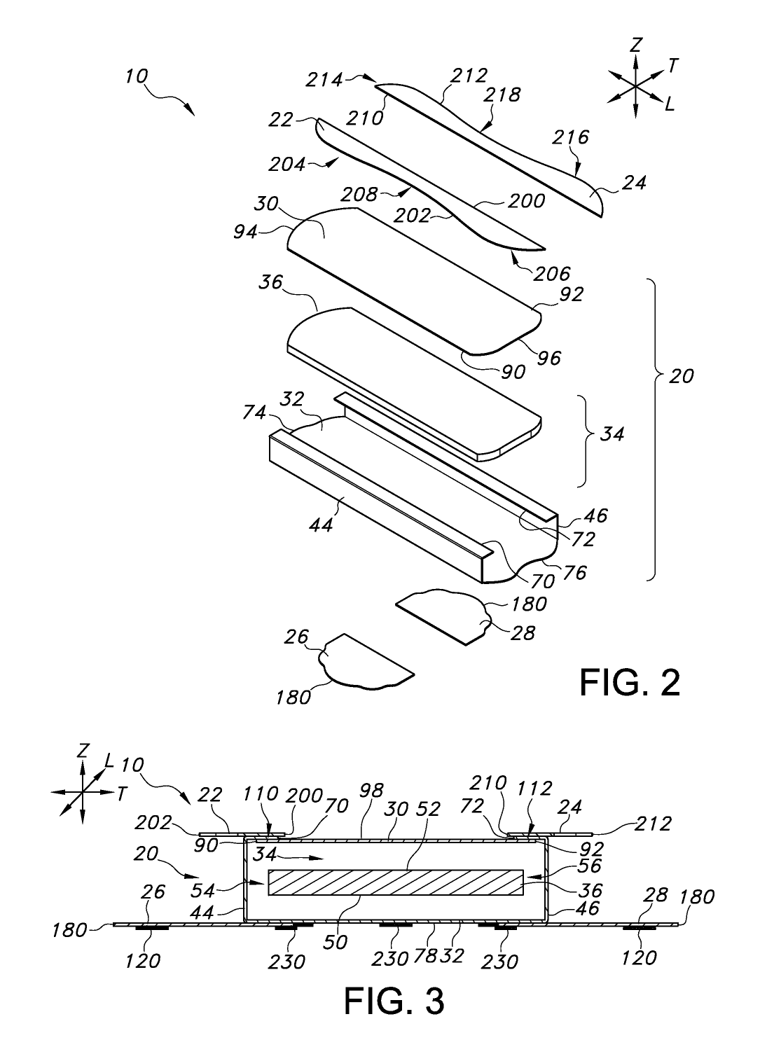

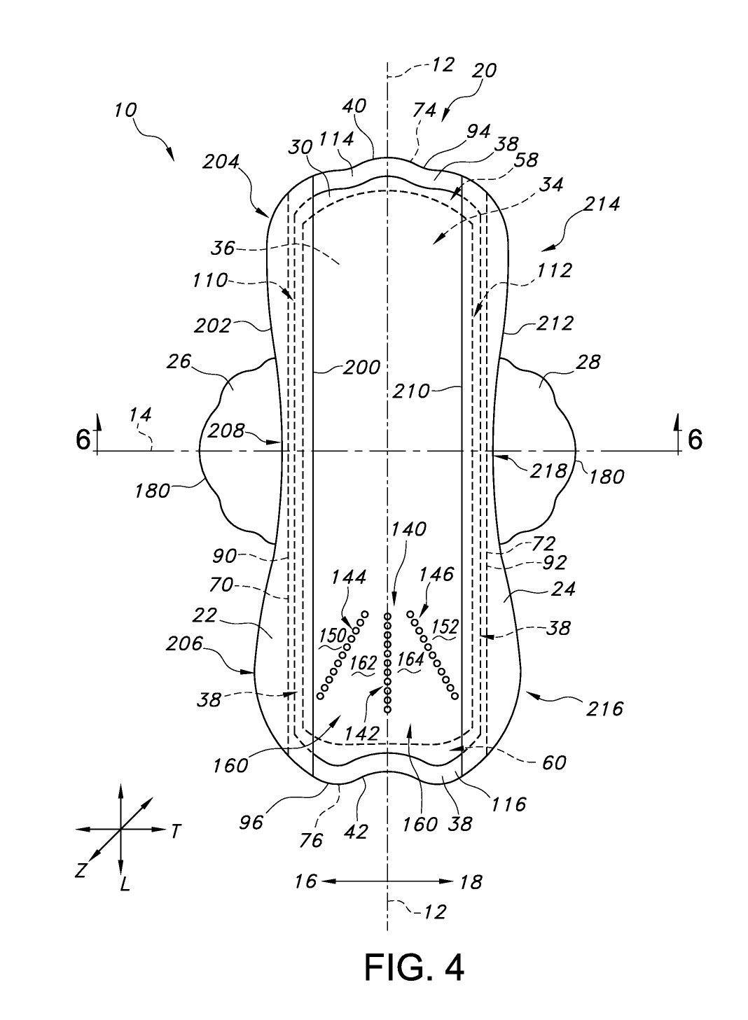

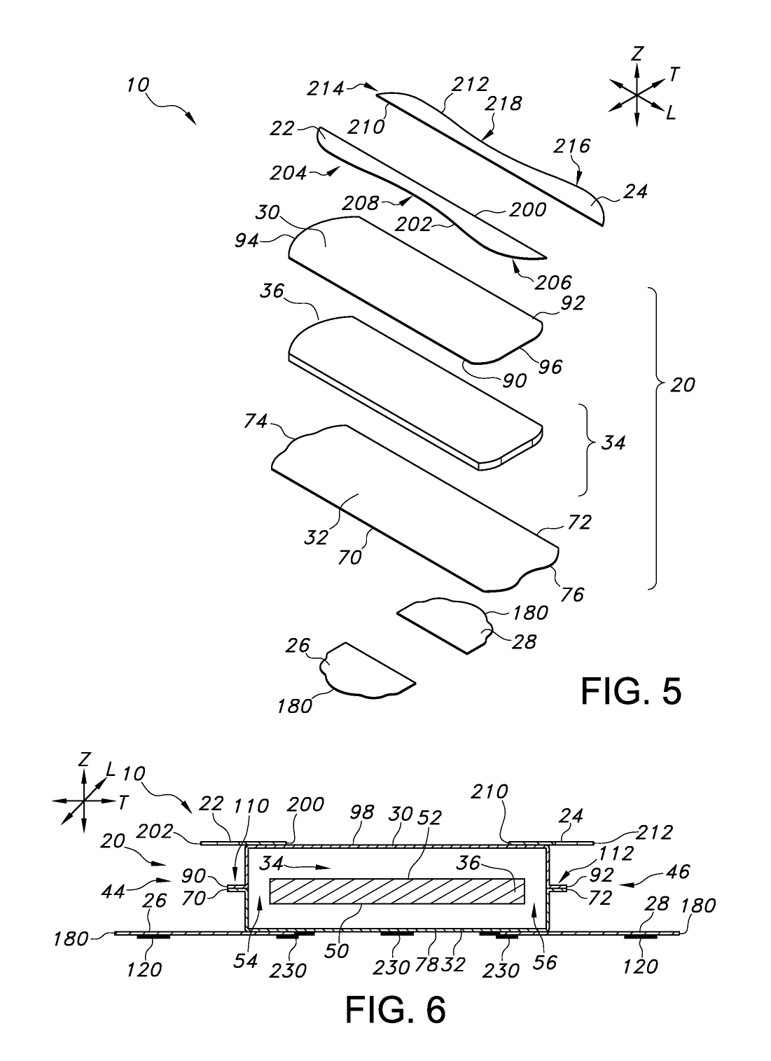

Referring to FIGS. 1-3, FIG. 1 provides an illustration of a top view of an embodiment of an exemplary absorbent article 10, FIG. 2 provides an illustration of an exploded perspective view of the absorbent article 10 of FIG. 1, and FIG. 3 provides an illustration of a cross-sectional view of the absorbent article 10 of FIG. 1 taken along line 3-3. Referring to FIGS. 4-6, FIG. 4 provides an illustration of a top view of a second embodiment of an exemplary absorbent article 10, FIG. 5 provides an illustration of an exploded perspective view of the absorbent article 10 of FIG. 4, and FIG. 6 provides an illustration of a cross-sectional view of the absorbent article 10 of FIG. 4 taken along line 6-6. The absorbent article 10 can have a longitudinal direction (L), a transverse direction (T), and a depth direction (Z). As illustrated in FIG. 1-6, the absorbent article 10 can have a longitudinal axis 12 and a transverse axis 14. The absorbent article 10 can have a chassis 20, a pair of non-integral side covers, 22 and 24, and a pair of non-integral wings, 26 and 28. The absorbent article 10 can be symmetrical about the longitudinal axis 12 of the absorbent article 10 and asymmetrical about the transverse axis 14 of the absorbent article 10.

The chassis 20 can have a wearer facing, liquid permeable topsheet layer 30 and a garment facing, liquid impermeable backsheet layer 32. An absorbent system 34 can be positioned between the topsheet layer 30 and the backsheet layer 32 and the absorbent system 34 can have at least an absorbent core 36. The topsheet layer 30 and the backsheet layer 32 can be bonded together to form a seal 38 which can contain the absorbent system 34 and body exudates received into the chassis through the topsheet layer 30. The bonding of the topsheet layer 30 and the backsheet layer 32 can be accomplished via any known bonding technique. For example, the topsheet layer 30 and the backsheet layer 32 can be bonded together by adhesive bonding, ultrasonic bonding, thermal bonding, heat press pattern bonding, or any other suitable bonding method known in the art.

The chassis 20 can have a first transverse direction end edge 40, a second transverse direction end edge 42 opposite the first transverse direction end edge 40, and a pair of opposing longitudinal direction side edges, 44 and 46. In various embodiments, the chassis 20 can take on various geometries but will generally have a pair of opposing longitudinal direction side edges, 44 and 46, and a pair of opposing transverse direction end edges 40 and 42. In various embodiments, the longitudinal direction side edges, 44 and 46, can be linear in the longitudinal direction (L) of the chassis 20. In various embodiments, the transverse direction end edges, 40 and 42, can be linear or can have at least one curve (convex and/or concave) in the transverse direction (T) of the chassis 20. In various embodiments, the longitudinal direction side edges, 44 and 46, can be linear in the longitudinal direction (L) of the chassis 20 and the transverse direction end edges, 40 and 42, can be linear in the transverse direction (T) of the chassis 20. In various embodiments, the longitudinal direction side edges, 44 and 46, can be linear in the longitudinal direction (L) of the chassis 20 and the transverse direction end edges, 40 and 42, can have at least one curve (convex and/or concave) in the transverse direction (T) of the chassis 20. The chassis 20 can be symmetrical about the longitudinal axis 12 of the absorbent article 10 and asymmetrical about the transverse axis 14 of the absorbent article 10.

The absorbent system 34 can have a garment facing surface 50, a wearer facing surface 52, an opposing pair of longitudinal direction peripheral regions, 54 and 56, and an opposing pair of transverse direction peripheral regions, 58 and 60. The garment facing surface 50 of the absorbent system 34 is the surface of the absorbent system 34 which is closest to the undergarment worn by the wearer. The wearer facing surface 52 of the absorbent system 34 is the surface of the absorbent system 34 which is closest to the body of the wearer. The absorbent system 34 can have at least an absorbent core 36 such as illustrated in FIGS. 1-6. In various embodiments, the absorbent system 34 can have at least one additional layer(s) such as, for example, but not limited to, a surge layer, a fluid intake layer, a transfer delay layer, and/or a distribution layer. It is to be understood that the absorbent system 34 can have any combination of layer(s) deemed suitable. The layer(s) of the absorbent system 34 can be present in any order deemed suitable in the depth direction (Z) of the absorbent article 10 between the topsheet layer 30 and the backsheet layer 32.

In various embodiments, such as, for example, illustrated in FIGS. 1-3, the backsheet layer 32 can overlay the garment facing surface 50 of the absorbent system 34, each of the longitudinal direction peripheral regions, 54 and 56, of the absorbent system 34, and a least a portion of the wearer facing surface 52 of the absorbent system 34. In such embodiments, the backsheet layer 32 can partially enclose the absorbent system 34 by wrapping around the absorbent system 34 until the longitudinal direction peripheral edges, 70 and 72, of the backsheet layer 32 overlay a portion of the wearer facing surface 52 of the absorbent system 34. In such embodiments, the portions of the backsheet layer 32 which overlay the longitudinal direction peripheral regions, 54 and 56, can form the longitudinal direction side edges, 44 and 46, of the chassis 20. In such embodiments, the topsheet layer 30 can overlay the wearer facing surface 52 of the absorbent system 34 and can partially enclose the absorbent system 34. In such embodiments, the longitudinal direction peripheral edges, 90 and 92, of the topsheet layer 30 and the longitudinal direction peripheral edges, 70 and 72, of the backsheet layer 32 can be bonded to each other forming longitudinal direction bond regions, 110 and 112. Each of the longitudinal direction bond regions, 110 and 112, overlay the wearer facing surface 52 of the absorbent system 34 and form a portion of the seal 38 between the topsheet layer 30 and the backsheet layer 32 of the chassis 20. In such embodiments, the absorbent system 34 can have transverse direction peripheral regions, 58 and 60, beyond which can extend portions of the topsheet layer 30 and backsheet layer 32. The transverse direction peripheral edges, 94 and 96, of the topsheet layer 30 can be bonded to the transverse direction peripheral edges, 74 and 76, of the backsheet layer 32 to form transverse direction bond regions, 114 and 116, respectively, which can form a portion of the seal 38 between the topsheet layer 30 and the backsheet layer 32 of the chassis 20. In such embodiments, the longitudinal direction bond regions, 110 and 112, and the transverse direction bond regions, 114 and 116, together form the seal 38 of the chassis 20 of the absorbent article 10.

In various embodiments, such as, for example, illustrated in FIGS. 4-6, the backsheet layer 32 can overlay a garment facing surface 50 of the absorbent system 34 and a portion of each of the longitudinal direction peripheral regions, 54 and 56, of the absorbent system 34. In such embodiments, the topsheet layer 30 can overlay the wearer facing surface 52 of the absorbent system 34 and a portion of each of the longitudinal direction peripheral regions, 54 and 56, of the absorbent system 34. In such embodiments, the longitudinal direction peripheral edges, 90 and 92, of the topsheet layer 30 and the longitudinal direction peripheral edges, 70 and 72, of the backsheet layer 32 can be bonded to each other in the vicinity of the longitudinal direction peripheral regions, 54 and 56, forming longitudinal direction bond regions, 110 and 112. Each of the longitudinal direction bond regions, 110 and 112, form a portion of the seal 38 between the topsheet layer 30 and the backsheet layer 32 of the chassis 20. In such embodiments, the absorbent article 34 can have transverse direction peripheral regions, 58 and 60, beyond which can extend portions of the topsheet layer 30 and the backsheet layer 32. The transverse direction peripheral edges, 94 and 96, of the topsheet layer 30 can be bonded to the transverse direction peripheral edges, 74 and 76, of the backsheet layer 32 to form transverse direction bond regions, 114 and 116, respectively, which can form a portion of the seal 38 between the topsheet layer 30 and the backsheet layer 32 of the chassis 20. In such embodiments, the longitudinal direction bond regions, 110 and 112, and the transverse direction bond regions, 114 and 116, together form the seal 38 of the chassis 20 of the absorbent article 10. In various embodiments, the topsheet layer 30 and the backsheet layer 32 can be sized such that the longitudinal direction peripheral edges, 90 and 92, of the topsheet layer 30 substantially align with the longitudinal direction peripheral edges, 70 and 72, of the backsheet layer 32 in the longitudinal direction bond regions, 110 and 112. In various embodiments, the topsheet layer 30 and the backsheet layer 32 can be sized such that the longitudinal direction peripheral edges, 90 and 92, of the topsheet layer 30 can be positioned closer to the longitudinal axis 12 of the absorbent article 10 than the longitudinal direction peripheral edges, 70 and 72, of the backsheet layer 32 in the longitudinal direction bond regions, 110 and 112. In various embodiments, the topsheet layer 30 and the backsheet layer 32 can be sized such that the longitudinal direction peripheral edges, 90 and 92, of the topsheet layer 30 can be positioned further from the longitudinal axis 12 of the absorbent article 10 than the longitudinal direction peripheral edges, 70 and 72, of the backsheet layer 32 in the longitudinal direction bond regions, 110 and 112.

In various embodiments, the topsheet layer 30 can overlay the wearer facing surface 52 of the absorbent system 34, each of the longitudinal direction peripheral regions, 54 and 56, of the absorbent system 34, and at least a portion of the garment facing surface 50 of the absorbent system 34. In such embodiments, the topsheet layer 30 can partially enclose the absorbent system 34 by wrapping around the absorbent system 34 until the longitudinal direction peripheral edges, 90 and 92, of the topsheet layer 30 overlay a portion of the garment facing layer 32 of the absorbent system 34. In such embodiments, the portions of the topsheet layer 30 which overlay the longitudinal direction peripheral regions, 54 and 56, can form the longitudinal direction side edges, 44 and 46, of the chassis 20. In such embodiments, the backsheet layer 32 can overlay the garment facing surface 50 of the absorbent system 34 and can partially enclose the absorbent system 34. In such embodiments, the longitudinal direction peripheral edges, 90 and 92, of the topsheet layer 30 and the longitudinal direction peripheral edges, 70 and 72, of the backsheet layer 32 can be bonded to each other forming longitudinal direction bond regions, 110 and 112. Each of the longitudinal direction bond regions, 110 and 112, overlay the garment facing surface 50 of the absorbent system 34 and form a portion of the seal 38 between the topsheet layer 30 and the backsheet layer 32 of the chassis 20. In such embodiments, the absorbent system 34 can have transverse direction peripheral regions, 58 and 60, beyond which can extend portions of the topsheet layer 30 and backsheet layer 32. The transverse direction peripheral edges, 94 and 96, of the topsheet layer 30 can be bonded to the transverse direction peripheral edges, 74 and 76, of the backsheet layer 32 to form transverse direction bond regions, 114 and 116, respectively, which can form a portion of the seal 38 between the topsheet layer 30 and the backsheet layer 32 of the chassis 20. In such embodiments, the longitudinal direction bond regions, 110 and 112, and the transverse direction bond regions, 114 and 116, together form the seal 38 of the chassis 20 of the absorbent article 10.

The absorbent article 10 can have a pair of opposing non-integral side covers, 22 and 24. Each of the non-integral side covers, 22 and 24, can extend in the longitudinal direction (L) and can extend the length of the chassis 20 of the absorbent article 10 from the first transverse direction end edge 40 of the chassis 20 to the second transverse direction end edge 42 of the chassis 20. In various embodiments, such as, for example, illustrated in FIGS. 1-3, the non-integral side covers, 22 and 24, can be bonded to an exterior surface 78 of the backsheet layer 32. In such embodiments, the side covers, 22 and 24, can be positioned over the longitudinal direction bond regions, 110 and 112, which form a portion of the seal 38 of the chassis 20 and which are positioned over the wearer facing surface 52 of the absorbent system 34. In such a positioning, the side covers, 22 and 24, can provide a softer and less irritating material to be in contact with the skin of the wearer than the exterior surface 78 of the backsheet layer 32. In such a positioning, the side covers, 22 and 24, can prevent the longitudinal direction bond regions, 110 and 112, from coming into direct contact with the skin of the wearer. In such a positioning, when the wings, 26 and 28, of the absorbent article 10 are wrapped about a wearer's undergarment, the side covers, 22 and 24, which are independent from the wings, 26 and 28, can remain undistorted by the folding of the wings, 26 and 28, about the wearer's undergarment. In various embodiments, such as, for example, illustrated in FIGS. 4-6, the non-integral side covers, 22 and 24, can be bonded to the wearer facing surface 98 of the topsheet layer 30. In such embodiments, the side covers, 22 and 24, can be positioned over the longitudinal direction bond regions, 110 and 112, which form a portion of the seal 38 of the chassis 20 and which are located in the vicinity of the longitudinal direction peripheral regions, 54 and 56. In such a positioning, the side covers, 22 and 24, can provide a softer and less irritating material to be in contact with the skin of the wearer than the seal 38 of the chassis 20. In such a positioning, when the wings, 26 and 28, of the absorbent article 10 are wrapped about a wearer's undergarment, the side covers, 22 and 24, which are independent from the wings, 26 and 28, can remain undistorted by the folding of the wings, 26 and 28, about the wearer's undergarment. In an embodiment, the non-integral side covers, 22 and 24, can be bonded to the wearer facing surface 98 of the topsheet layer 30 in a position overlaying the wearer facing surface 52 of the absorbent system 34 and can be positioned over the longitudinal direction bond regions, 110 and 112, which form a portion of the seal 38 of the chassis 20 and which overlay the garment facing surface 50 of the absorbent system 34. In such a positioning, when the wings, 26 and 28, of the absorbent article 10 are wrapped about a wearer's undergarment, the side covers, 22 and 24, which are independent from the wings, 26 and 28, can remain undistorted by the folding of the wings, 26 and 28, about the wearer's undergarment.

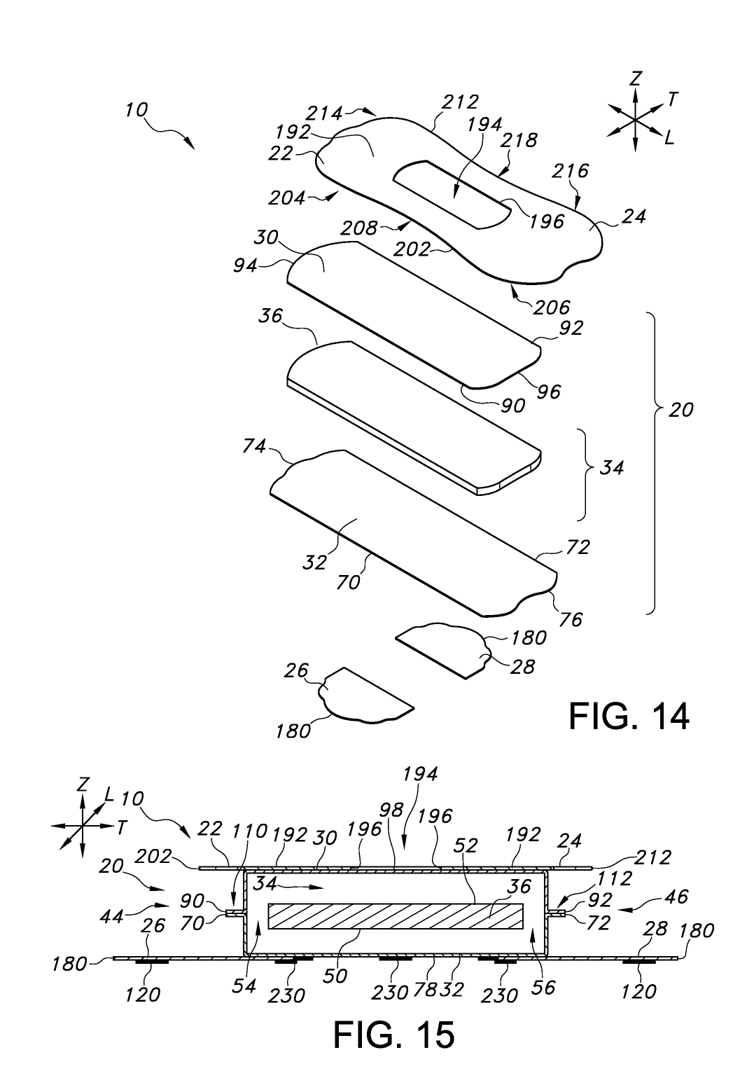

In various embodiments, the absorbent article 10 can have a pair of non-integral wings, 26 and 28, extending outwardly, in the transverse direction (T), from the absorbent article 10. The wings, 26 and 28, can be bonded to the exterior surface 78 of the backsheet layer 32 of the chassis 20 of the absorbent article 10. The wings, 26 and 28, can drape over the edges of the wearer's undergarment so that the wings, 26 and 28, are disposed between the edges of the wearer's undergarment and her thighs. The wings, 26 and 28, can serve at least two purposes. First, the wings, 26 and 28, can prevent soiling of the wearer's undergarment by forming a barrier along the edges of the undergarment. Second, each wing, 26 and 28, can be provided with an attachment aid 120, such as, for example, a garment attachment adhesive or a hook, to keep the absorbent article 10 securely and properly positioned in the undergarment. The wings, 26 and 28, can wrap around the crotch region of the wearer's undergarment to aid in securing the absorbent article 10 to the wearer's undergarment when in use. Each wing, 26 and 28, can fold under the crotch region of the wearer's undergarment and the attachment aid 120 can either form a secure attachment to the opposite wing, 26 or 28, or directly to the surface of the wearer's undergarment. In various embodiments, the wings, 26 and 28, can be constructed of materials similar to the topsheet layer 20, the backsheet layer 22 or combinations of these materials. In various embodiments, the wings, 26 and 28, can be separate components which can each individually be bonded to the backsheet layer 32. In various embodiments, the wings, 26 and 28, can be extensions from, and integral with, a wing bridge 190 (shown in FIG. 14) which can be bonded to the backsheet layer 32 and which can extend across the transverse direction (T) width of the absorbent article 10.

Each of these components of the absorbent article 10, as well as additional components, will be described in more detail herein.

### Chassis:

As described herein, the chassis 20 of an absorbent article 10 can have a topsheet layer 30, a backsheet layer 32 and an absorbent system 34 positioned between the topsheet layer 20 and the backsheet layer 32. The absorbent system 34 can have at least an absorbent core 36. In various embodiments, the absorbent system 34 can have at least one additional layer such as a surge layer 62, a fluid intake layer 64, a transfer delay layer, and/or a distribution layer. The additional layer(s) can be present in any combination deemed suitable.

### Topsheet Layer:

The topsheet layer 30 defines a wearer facing surface 98 of the absorbent article 10 that may directly contact the body of the wearer and is liquid permeable to receive body exudates. The topsheet layer 30 is desirably provided for comfort and conformability and functions to direct body exudates away from the body of the wearer, through its own structure, and towards the absorbent system 34. The topsheet layer 30 desirably retains little to no liquid in its structure, so that it provides a relatively comfortable and non-irritating surface next to the skin of the wearer of the absorbent article 10.

The topsheet layer 30 can be a single layer of material, or alternatively, can be multiple layers that have been laminated together. The topsheet layer 30 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 30.

In various embodiments, the topsheet layer 30 can be constructed from various nonwoven webs such as meltblown webs, spunbond webs, hydroentangled spunlace webs, or through air bonded carded webs. Examples of suitable topsheet layer 30 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 30 can be a bonded carded web made of polypropylene and polyethylene such as that obtainable from Sandler Corporation, Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 30, each of which is hereby incorporated by reference thereto in its entirety. Additional topsheet layer 30 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 30 may contain a plurality of apertures (not shown) formed therethrough to permit body exudates to pass more readily into the absorbent system 34. The apertures may be randomly or uniformly arranged throughout the topsheet layer 30. The size, shape, diameter, and number of apertures may be varied to suit an absorbent article's 10 particular needs.

In various embodiments, the topsheet layer 30 can have a basis weight ranging from about 5, 10, 15, 20 or 25 gsm to about 50, 100, 120, 125 or 150 gsm. For example, in an embodiment, a topsheet layer 30 can be constructed from a through air bonded carded web having a basis weight ranging from about 15 gsm to about 100 gsm. In another example, a topsheet layer 30 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics and others.

In various embodiments, the topsheet layer 30 can be at least partially hydrophilic. In various embodiments, a portion of the topsheet layer 30 can be hydrophilic and a portion of the topsheet layer 30 can be hydrophobic. In various embodiments, the portions of the topsheet layer 30 which can be hydrophobic can be either an inherently hydrophobic material or can be a material treated with a hydrophobic coating.

### Absorbent System:

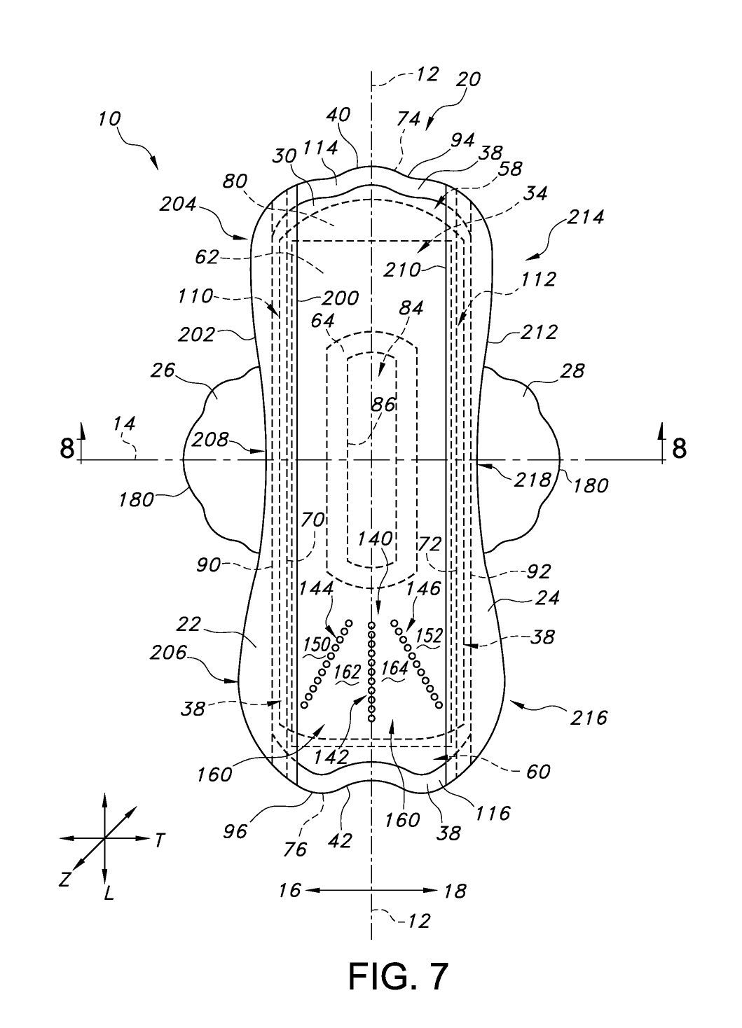

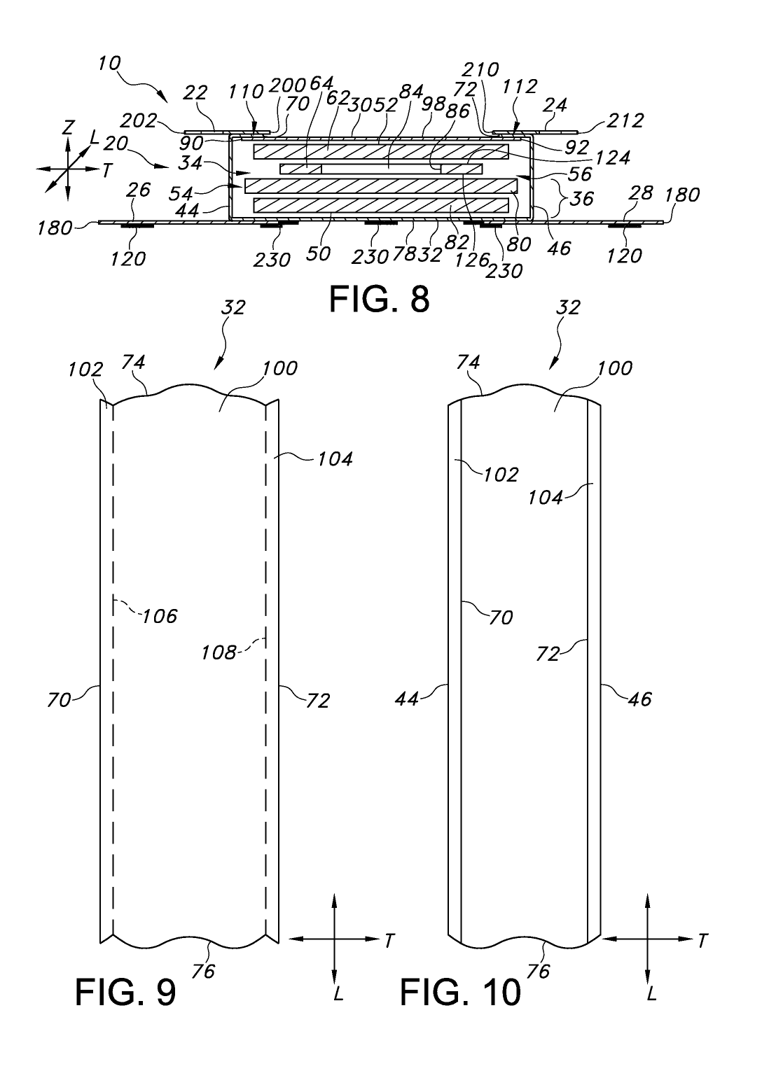

The absorbent system 34 can have a garment facing surface 50, a wearer facing surface 52, an opposing pair of longitudinal direction peripheral regions, 54 and 56, and an opposing pair of transverse direction peripheral regions, 58 and 60. The garment facing surface 50 of the absorbent system 34 is the surface of the absorbent system 34 which is closest to the undergarment worn by the wearer. The wearer facing surface 52 of the absorbent system 34 is the surface of the absorbent system 34 which is closest to the body of the wearer. The absorbent system 34 can have at least an absorbent core 36 such as illustrated in FIGS. 1-6. In various embodiments, the absorbent core 36 can be a single layer construction such as illustrated in FIGS. 1-6. In various embodiments, the absorbent core 36 can have at least two layers of material, such as a wearer facing layer 80 and a garment facing layer 82 such as illustrated in FIGS. 7 and 8. In various embodiments, the absorbent system 34 can have additional layers such as, for example, but not limited to, a surge layer, a fluid intake layer, a transfer delay layer, and/or a distribution layer. FIGS. 7 and 8 illustrate an embodiment of an absorbent article 10 having an absorbent system 34 which can have an absorbent core 36 having two layers of material, 80 and 82, a surge layer 62 and a fluid intake layer 64. It is to be understood that the absorbent system 34 can have any combination of layer(s) (e.g., a surge layer, a fluid intake layer, a transfer delay layer, an absorbent core, and/or a distribution layer) as well any additional layer deemed suitable. The layer(s) of the absorbent system 34 can be present in any order deemed suitable in the depth direction (Z) of the absorbent article 10 between the topsheet layer 30 and the backsheet layer 32.

In an embodiment, the absorbent system 34 can have an absorbent core 36 which can be a single layer construction. In an embodiment, the absorbent system 34 can have an absorbent core 36 which can be a single layer construction and the absorbent system 34 can have at least one, two, three or four additional layer(s) which can be selected from a surge layer 62, a fluid intake layer 64, a transfer delay layer, and/or a distribution layer. In an embodiment, the absorbent system 32 can have an absorbent core 36 which can be of a two layer construction, such as having a wearer facing layer 80 and a garment facing layer 82. In an embodiment, the absorbent system 34 can have an absorbent core 36 which can have at least two layers of material, such as a wearer facing layer 80 and a garment facing layer 82, and the absorbent system 34 can have at least one, two, three, or four additional layer(s) which can be selected from a surge layer 62, a fluid intake layer 64, a transfer delayer layer, and/or a distribution layer.

### Absorbent Core:

The absorbent system 34 can have an absorbent core 36 positioned between the topsheet layer 30 and the backsheet layer 32, such as illustrated in FIGS. 1-6. The absorbent core 36 can generally be any single layer structure or combination of layer components, which can demonstrate some level of compressibility, conformability, be non-irritating to a wearer's skin, and capable of absorbing and retaining liquids and other body exudates. Additionally, the absorbent core 36 can provide additional capacity to absorb and retain body exudates such as menses. In various embodiments, the absorbent core 36 can be formed from a variety of different materials and can contain any number of desired layers. For example, the absorbent core 36 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of a wood pulp fluff can be identified with the trade designation NB 416, available from Weyerhaeuser Corp., and is a bleached, highly absorbent wood pulp containing primarily soft wood fibers.

In various embodiments, if desired, the absorbent core 36 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and a-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 36 in any amount as desired.

Regardless of the combination of absorbent materials used in the absorbent core 36, the absorbent materials can be formed into a web structure by employing various conventional methods and techniques. For example, the absorbent web can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art.

The shape of the absorbent core 36 can vary as desired and can comprise any one of various shapes including, but not limited to, triangular, rectangular, dog-bone and elliptical shapes. In various embodiments, the absorbent core 36 can have a shape that generally corresponds with the overall shape of the chassis 20. The dimensions of the absorbent core 36 can be substantially similar to those of the chassis 20, however, it will be appreciated that the dimensions of the absorbent core 36 while similar, will often be less than those of the overall chassis 20, in order to be adequately contained therein.

By way of example, suitable materials and/or structures for the absorbent core 36 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al., each of which is hereby incorporated by reference thereto in its entirety.

As described above, in various embodiments, an absorbent core 36 can be a single layer structure and can include, for example, a matrix of cellulosic fluff and superabsorbent material. In various embodiments, an absorbent core 36 can have at least two layers of material, such as, for example, a wearer facing layer 80 and a garment facing layer 82, such as illustrated in FIGS. 7 and 8. In various embodiments, the two layers, 80 and 82, can be identical to each other. In various embodiments, the two layers, 80 and 82, can be different from each other. In such embodiments, the two layers, 80 and 82, can provide the chassis 20 with different absorption properties as deemed suitable. In various embodiments, the wearer facing layer 80 of the absorbent core 36 may be constructed of an airlaid material and the garment facing layer 82 of the absorbent core 36 may be constructed of a superabsorbent polymer-containing compressed sheet. In such embodiments, the airlaid material can have a basis weight from about 40 to about 200 gsm and the superabsorbent polymer-containing compressed sheet can be a cellulosic fluff based material that can be a combination of cellulosic pulp and SAP enclosed with a tissue carrier and having a basis weight from about 40 to about 400 gsm.

### Additional Absorbent System Layers:

As described herein, the absorbent system 34 can have at least an absorbent core 36 and, in various embodiments, the absorbent system 34 can have at least one addition layer. The at least one additional layer can be at least one of a surge layer 62, a fluid intake layer 64, a transfer delay layer, and/or a distribution layer. FIGS. 7 and 8 provide an exemplary illustration of an absorbent system 34 which has a surge layer 62, a fluid intake layer 64, and an absorbent core 36 which has a wearer facing layer 80 and a garment facing layer 82. The absorbent article 10 illustrated in the Figures is non-limiting and additional arrangements of layer(s) in the absorbent system 34 are contemplated herein.

### Surge Layer:

In various embodiments, an absorbent system 34 can include a surge layer 62 positioned between the topsheet layer 30 and the absorbent core 36. A surge layer 62 can be constructed of any woven or nonwoven material that is easily penetrated by body exudates. The surge layer 62 can help to absorb, decelerate, and diffuse surges or gushes of liquid that may be rapidly introduced into the chassis 20. The surge layer 62 can rapidly accept and temporarily hold the liquid prior to releasing the liquid into, for instance, the absorbent core 36 or any other layer of the absorbent system 34. Various woven fabrics and nonwoven webs can be used to construct the surge layer 62. For example, the surge layer 62 can comprise a nonwoven fabric layer composed of a meltblown or spunbond web of polyolefin or polyester filaments. Such nonwoven fabric layers may include conjugate, biconstituent and homopolymer fibers of staple or other lengths and mixtures of such fibers with other types of fibers. The surge layer 62 can also be a bonded card web or an airlaid web composed of natural and/or synthetic fibers. The bonded carded web may, for example, be a powder bonded carded web, an infrared bonded carded web, or a through air bonded carded web. The bonded carded webs can optionally include a mixture or blend of different fibers. The surge layer 62 can typically have a basis weight of less than about 100 gsm, and in some embodiments, from about 10 gsm to about 40 gsm.

### Fluid Intake Layer:

In various embodiments, the absorbent system 34 can include a liquid permeable fluid intake layer 64. Such a fluid intake layer 64 can be made of a material that can be capable of rapidly transferring, in the Z-direction, body exudates that are delivered to the topsheet layer 30. The fluid intake layer 64 can generally have any shape and/or size desired. In an embodiment, the fluid intake layer 64 can have a curved rectangular shape, with a length equal to or less than the overall length of the chassis 20, and a width less than the width of the chassis 20. For example, the fluid intake layer 64 can have a length of between about 20, 40 or 60 mm to about 150, 150, 175, 200 or 300 mm and a width of between about 10, 15 or 20 mm to about 60, 80 or 100 mm may be utilized. The fluid intake layer 64 can have a thickness in the depth direction (Z) from about 0.5 mm to about 3 mm. Any of a variety of different materials can be capable of being used for the fluid intake layer 64 to accomplish the above-mentioned functions. The material may be synthetic, cellulosic, or a combination of synthetic and cellulosic materials. The fluid intake layer 64 can be constructed from any woven or nonwoven material. For example, the fluid intake layer 64 can be constructed as an airlaid or TABCW material. For example, airlaid cellulosic tissues may be suitable for use in the fluid intake layer 64. The airlaid cellulosic tissue may have a basis weight ranging from about 10 or 100 gsm to about 250 or 300 gsm. The airlaid cellulosic tissue can be formed from hardwood and/or softwood fibers. An airlaid cellulosic tissue can have a fine pore structure and can provide an excellent wicking capacity, especially for menses.

Additionally, to further enhance the ability of the chassis 20 to transfer body exudates in the depth direction (Z) from the topsheet layer 30 toward any lower layers in the chassis 20 as well as to enhance the ability of the fluid intake layer 64 to conform to the wearer's body based on its ability to bend, the fluid intake layer 64 can have an opening 84 (shown in FIGS. 7 and 8) in the fluid intake layer 64 which can be any suitable shape, such as ovular, circular, rectangular, square, triangular, etc. In various embodiments, the opening 84 in the fluid intake layer 64 can be elongate and can be oriented in the longitudinal direction of the chassis 20. The opening 84 in the fluid intake layer 64 can be bounded by a perimeter 86 which can form an inner border or inner edge of the fluid intake layer 64. The opening 84 can extend in the depth direction (Z) of the fluid intake layer 64 from an upper, wearer facing surface 124 of the fluid intake layer 64 through and to a lower, garment facing surface 126 of the fluid intake layer 64.

The opening 84 can be located at various positions along the longitudinal and transverse axes of the fluid intake layer 64 depending upon the primary location of body exudate intake or the purpose for which the absorbent article 10 is being used. For example, in various embodiments, the fluid intake layer 64 and the opening 84 in the fluid intake layer 64 can be positioned so that it is in substantial alignment with the longitudinal axis 12 and the transverse axis 14 of the absorbent article 10. This allows the opening 84 to be centrally disposed so that it can be positioned below the main point of body exudate discharge and so that it can act as the primary body exudate receiving area for the absorbent article 10.

However, centralized positioning of the fluid intake layer 64 and the opening 84 of the fluid intake layer 64 is not required, and in various embodiments, depending on the primary location where body exudate intake might occur, the fluid intake layer 64 and the opening 84 of the fluid intake layer 64 may be substantially aligned with the longitudinal axis 12 only. Thus, in various embodiments, the fluid intake layer 64 and the opening 84 of the fluid intake layer 64 may be shifted in the longitudinal direction towards either transverse direction end edge, 40 or 42, of the chassis 20, so that the opening 84 of the fluid intake layer 64 is not in substantial alignment with the transverse axis 14 of the absorbent article 10.

The opening 84 in the fluid intake layer 64 can have a longitudinal length from about 15, 20, 30 or 50 mm to about 60, 75, 100 or 150 mm and can have a transverse width from about 10, 15, 20 or 30 mm to about 40, 60 or 80 mm. The opening 84 in the fluid intake layer 64 can be defined by the perimeter 86 and can have a length that is from about 15, 20 or 25% to about 70, 75, or 80% of the overall longitudinal length of the fluid intake layer 64 in the longitudinal direction. The opening 84 in the fluid intake layer 64 can be defined by the perimeter 86 and can have a width that can be from about 20, 25 or 30% to about 70, 75 or 80% of the overall width of the fluid intake layer 64 in the transverse direction. The opening 84 in the fluid intake layer 64 can serve to funnel and direct body exudates from the topsheet layer 30 and towards lower layers of the chassis 20 in the depth direction (Z). The opening 84 in the fluid intake layer 64 can also form a cup or well-like structure for holding body exudates and preventing leakage away from a central region of the absorbent article 10 and towards the edges of the absorbent article 10.

### Transfer Delay Layer:

In various embodiments, an absorbent system 34 can include a liquid permeable transfer delay layer (not shown) positioned between the topsheet layer 30 and the absorbent core 36. The transfer delay layer may contain a material that is substantially hydrophobic. For example, the transfer delay layer may be a nonwoven fibrous web composed of relatively hydrophobic materials, such as polypropylene, polyethylene, polyester, or the like, and also may be composed of a blend of such materials. One example of a material suitable for the transfer delay layer can be a spunbond web composed of polypropylene, multi-lobal fibers. Further examples of suitable transfer delay layers can include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay layer are described in U.S. Pat. No. 4,798,603 to Meyer, et al. and U.S. Pat. No. 5,248,309 to Serbiak, et al., each of which are hereby incorporated by reference in its entirety.

The transfer delay layer may generally have any size, such as a length of about 150 mm to about 300 mm. Typically, the length of the transfer delay layer can be approximately equal to the length of the chassis 20. The width of the transfer delay layer can be from between about 50 mm to about 75 mm. The transfer delay layer can have a basis weight less than about 250 gsm, and in some embodiments, between about 40 gsm and about 200 gsm.

### Distribution Layer:

In various embodiments, the absorbent system 34 can have a distribution layer (not shown) positioned below the absorbent core 36 and between the absorbent core 36 and the backsheet layer 32. The distribution layer can increase absorbency of the chassis 20. The distribution layer can be constructed of various materials such as, but not limited to, hydroentangled webs, through air bonded carded webs, meltblown webs, and meltblown microfiber webs. The distribution layer can include a hydrophilic material. The distribution layer can be smaller in size than the absorbent core 36.

In various embodiments, the distribution layer can have a longitudinal length from about 80, 90, 100, 110, 120, 125 or 130 mm to about 135, 140, 150, 160, 170, 180 or 190 mm and can have a transverse width from about 30, 35 or 40 mm to about 45, 50, 55 or 60 mm. In various embodiments, the distribution layer can have a density of greater than about 0.1 grams per cubic centimeter. The density can be calculated utilizing the formula: density =basis weight (gsm)/thickness (mm)/1000. In various embodiments, the distribution layer can have a basis weight from about 10, 20, 25, 30 or 50 gsm to about 60, 70, 80, 90, 100, 120, 140, 150, 160, 180 or 200 gsm.

In various embodiments, the distribution layer can be a hydroentangled web. The hydroentangled web can include a hydroentangled spunbond material and a pulp material. The hydroentangled spunbond material can include a polypropylene material. The spunbond material can be present in an amount from about 10% or 15% to about 20% or 25% of the hydroentangled web. The pulp material can be present in an amount from about 75% or 80% to about 85% or 90% of the hydroentangled web. The hydroentangled web can have a basis weight from about 30 or 60 gsm to about 90 or 200 gsm. The basis weight of the hydroentangled web can be balanced with the desired flexibility of the absorbent article 10. In various embodiments, the distribution layer can include a bicomponent fluid distribution layer, which can increase absorbency by providing a high void space and may be made of a through air bonded carded web, having a basis weight, in an embodiment, of between about 25 gsm and 100 gsm. In various embodiments, the distribution layer can be a meltblown microfiber web of polypropylene material and can have a basis weight from about 10 or 20 gsm to about 30, 50 or 100 gsm. In various embodiments, the meltblown microfiber web can be treated with wetting agents for adequate handling of body exudates. Examples of wetting agents can include, but are not limited to, surface active agents (or surfactants) having a hydrophilic lipophilic balance (HLB) of at least 6, 7 or 18. A variety of surfactants can be used and can include, but are not limited to, anionic, cationic, or neutral from a charge standpoint. Mixtures of surfactants and other wetting agents can also be used. A wetting agent add-on can range from about 0.1 or 0.2% to about 5 or 10%. In various embodiments, an add-on amount can be higher than 10%. For example, the meltblown microfiber web can be treated to impart hydrophlicity by either Aerosol GPG of Cytec or Ahcovel Base N-62 for example. Such material is available from Yuhan-Kimberly Ltd., Seoul, Korea and FlberTex, Malaysia.

### Backsheet Layer:

The backsheet layer 32 is generally liquid impermeable and is the portion of the chassis 20 which faces the garment of the wearer. The backsheet layer 32 can permit the passage of air or vapor out of the chassis 20 while still blocking the passage of liquids. Any liquid impermeable material may generally be utilized to form the backsheet layer 32. The backsheet layer 32 can be composed of a single layer or multiple layers, and these one or more layers can themselves comprise similar or different materials. Suitable material that may be utilized can be a microporous polymeric film, such as a polyolefin film of polyethylene or polypropylene, nonwovens and nonwoven laminates, and film/nonwoven laminates. The particular structure and composition of the backsheet layer 32 can be selected from various known films and/or fabrics with the particular material being selected as appropriate to provide the desired level of liquid barrier, strength, abrasion resistance, tactile properties, aesthetics and so forth. In various embodiments, a polyethylene film can be utilized that can have a thickness in the range of from about 0.2 or 0.5 mils to about 3.0 or 5.0 mils. An example of a backsheet layer 32 can be a polyethylene film such as that obtainable from Pliant Corporation, Schaumburg, Ill., USA. Another example can include calcium carbonate-filled polypropylene film. In still another embodiment, the backsheet layer 32 can be a hydrophobic nonwoven material with water barrier properties such as a nonwoven laminate, an example of which can be a spunbond, meltblown, meltblown, spunbond, four-layered laminate. The backsheet layer 32 can, therefore, be of a single or multiple layer construction, such as of multiple film layers or laminates of film and nonwoven fibrous layers. Suitable backsheet layers 32 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

Referring to FIGS. 1-3, 7 and 8, in various embodiments, the backsheet layer 32 can overlay a garment facing surface 50 of the absorbent system 34, each of the longitudinal direction peripheral regions, 54 and 56, of the absorbent system 34, and at least a portion of the wearer facing surface 52 of the absorbent system 34. In such embodiments, the backsheet layer 32 can partially enclose the absorbent system 34 by wrapping around the absorbent system 34 until the longitudinal direction peripheral edges, 70 and 72, of the backsheet layer 32 overlay a portion of the wearer facing surface 52 of the absorbent system 34. FIGS. 9 and 10 provide illustrations of an exemplary backsheet layer 32 in an unfolded and folded configuration, respectively. Referring to FIG. 9, the backsheet layer 32 can be in an unfolded and laid flat configuration. The backsheet layer 32 can have a central portion 100 and a pair of opposing longitudinal direction side portions, 102 and 104. The backsheet layer 32, in an unfolded and laid flat configuration can have a width greater than the width of the topsheet layer 30 of the chassis 20. In various embodiments, the side portions, 102 and 104, can each have transverse direction end edges which can correspond in a mirror-image relationship to the configuration of the transverse direction end edges, 74 and 76, of the central portion 100 of the backsheet layer 32. Having a mirror-image correspondence between the transverse direction end edges of the side portions, 102 and 104, and the transverse direction end edges, 74 and 76, of the central portion 100 of the backsheet layer 32 when the backsheet layer 32 is in a flat, unfolded configuration can allow for each of the transverse direction end edges of the side portions, 102 and 104, to correspond to and substantially align with the transverse direction end edges, 74 and 76, of the central portion 100 of the backsheet layer 32 when the backsheet layer 32 is in a folded configuration. As illustrated in FIG. 9, each of the transverse direction end edges, 74 and 76, of the central portion 100 of the backsheet layer 32 has a curvature and the transverse direction end edges of each of the side portions, 102 and 104, have the mirror-image curvature which can correspond with the transverse direction end edges, 74 and 76, when the backsheet layer 32 is in a folded configuration, such as illustrated in FIG. 10.

In the manufacture of the chassis 20 for the absorbent article 10 such as illustrated in FIGS. 1-3, 7 and 8, central portion 100 of the backsheet layer 32 can overlay a garment facing surface 50 of the absorbent system 34 and the side portions, 102 and 104, can be re-positioned to wrap around the absorbent system 34 such that the side portions, 102 and 104, can overlay each of the longitudinal direction peripheral regions, 54 and 56, of the absorbent system 34 and the longitudinal direction peripheral edges, 70 and 72, of the backsheet layer 32 can be positioned over a wearer facing surface 52 of the absorbent system 34. The side portions, 102 and 104, can begin to wrap around an absorbent system in the fold areas, 106 and 108. It is to be understood that the fold areas, 106 and 108, can be, but need not be, a single linear fold line in the backsheet layer 32. FIG. 10 provides an illustration of an exemplary embodiment in which the side portions, 102 and 104, of the backsheet layer 32 have been re-positioned to overlay an absorbent system 34 (not shown in FIG. 10). The longitudinal direction peripheral edges, 70 and 72, of the backsheet layer 32 can then be bonded to the longitudinal direction peripheral edges, 90 and 92, of the topsheet layer 30 to partially enclose the absorbent system 34 and to form a portion of the seal 38 of the chassis 20 of the absorbent article 10.

Referring to FIGS. 4-6, in various embodiments, the backsheet layer 32 can overlay a garment facing surface 50 of the absorbent system 34 and a portion of each of the longitudinal direction peripheral regions, 54 and 56, of the absorbent system 34. In such embodiments, the topsheet layer 30 can overlay the wearer facing surface 52 of the absorbent system 34 and a portion of each of the longitudinal direction peripheral regions, 54 and 56, of the absorbent system 34. In such embodiments, the longitudinal direction peripheral edges, 90 and 92, of the topsheet layer 30 and the longitudinal direction peripheral edges, 70 and 72, of the backsheet layer 32 can be bonded to each other forming longitudinal direction bond regions, 110 and 112. Each of the longitudinal direction bond regions, 110 and 112, form a portion of the seal 38 between the topsheet layer 30 and the backsheet layer 32 of the chassis 20. In such embodiments, the absorbent article 34 can have transverse direction peripheral regions, 58 and 60, beyond which can extend portions of the topsheet layer 30 and the backsheet layer 32. The transverse direction peripheral edges, 94 and 96, of the topsheet layer 30 can be bonded to the transverse direction peripheral edges, 74 and 76, of the backsheet layer 32 to form transverse direction bond regions, 114 and 116, respectively, which can form a portion of the seal 38 between the topsheet layer 30 and the backsheet layer 32 of the chassis 20. In such embodiments, the longitudinal direction bond regions, 110 and 112, and the transverse direction bond regions, 114 and 116, together form the seal 38 of the chassis 20 of the absorbent article 10.

### Nesting Edges:

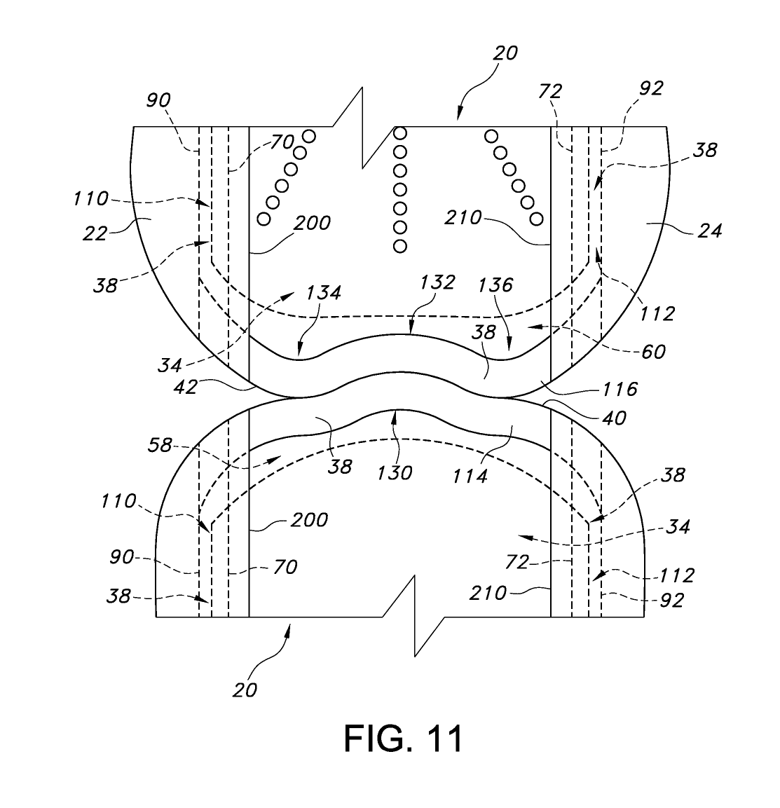

In various embodiments, the transverse direction end edges, 40 and 42, of the chassis 20 can be linear or can have at least one curve (convex and/or concave) in the transverse direction (T) of the chassis 20. In various embodiments, one of the transverse direction end edges, 40 or 42, can have at least one curve (convex and/or concave) and the other of the transverse direction end edges, 40 or 42, can have at least one curve (convex and/or concave) and the curve of the first transverse direction end edge, 40 or 42, can be positioned into a nesting configuration with the second of the transverse direction end edge, 40 or 42. A curve can be defined as convex or concave when viewed from the perspective of the intersection of the longitudinal axis 12 and the transverse axis 14. FIG. 11 provides a sectional view of an absorbent article 10 in which the transverse direction end edges, 40 and 42, can be positioned into a nesting configuration with each other. As illustrated in FIG. 11, transverse direction end edge 40 can have a convex curve 130 and transverse direction end edge 42 can have a concave curve 132 and two convex curves, 134 and 136. The convex curve 130 of transverse direction end edge 40 can nest between the two convex curves, 134 and 136, of transverse direction end edge 42. The nesting configuration can also position convex curve 130 of transverse direction end edge 40 into an abutment with concave curve 132 of transverse direction end edge 42.

### Flexure Features:

In various embodiments, the chassis 20 can have at least one flexure feature 140. The at least one flexure feature 140 can be positioned in chassis 20 in a location intended to be worn towards the posterior of the wearer. The relative position of the at least one flexure feature 140 can be seen in FIGS. 1, 4 and 7. The at least one flexure feature 140 can help initiate and influence shaping of the absorbent article 10 into a raised and tented configuration. The raised and tented configuration can conform to the gluteal cleft and can move in response to the alternating movement of the wearer's legs thereby helping the absorbent article 10 stay in place for leakage protection and comfort. In various embodiments, the at least one flexure feature 140 can create different bend resistances across the chassis 20 of the absorbent article 10. The at least one flexure feature 140 can be created by physical discontinuities in the chassis 20 and/or elements of the chassis 20. For example, the at least one flexure feature 140 can be created by pre-folding, scoring, indenting, perforating, embossing, bonding, or combinations thereof. In various embodiments, the at least one flexure feature 140 can be created by scoring, folding, indenting, perforating, embossing, or bonding one or more layers of the chassis 20 of the absorbent article 10. In various embodiments, the at least one flexure feature 140 can be created with changes in elevation and/or density to the chassis 20 and/or elements of the chassis 20. Optionally, the at least one flexure feature 140 helps initiate and regulate dynamic movement in the posterior region of the absorbent article 10.

In various embodiments, the flexure feature 140 of the chassis 20 can have a first flexure 142 which may extend in a direction generally parallel to the longitudinal axis 12 of the chassis 20 of the absorbent article 10. In various embodiments, the flexure feature 140 of the chassis 20 can have a second flexure 144 which can be spaced transversely outward from the first flexure 142 in a first direction 16. The second flexure 144 can define a first side portion 150 of the chassis 20. The first side portion 150 is positioned transversely outward from the second flexure 144 in the first direction 16. In various embodiments, the flexure feature 140 of the chassis 20 can have a third flexure 146 which can be spaced transversely outward from the first flexure 142 in a second direction 18 which is opposite the first direction 16. The third flexure 146 can define a second side portion 152 of the chassis 20. The second side portion 152 is positioned transversely outward from the third flexure 146 in the second direction 18.

The second flexure 144 and the third flexure 146 can define a dynamic region 160 of the chassis 20 and of the absorbent article 10 there between. The second flexure 144 and the first flexure 142 define a first side 162 of the dynamic region 160. The third flexure 146 and the first flexure 142 define a second side 164 of the dynamic region 160.

In various embodiments, the second flexure 144 and/or the third flexure 146 can be oriented in a direction generally parallel to the longitudinal axis 12 (not shown). In various embodiments, the second flexure 144 and/or the third flexure 146 may be positioned at an angle relative to the longitudinal axis 12. For example, in various embodiments, the second flexure 144 and/or the third flexure 146 may diverge rearward at an angle relative to the longitudinal axis 12 which can be from about 1, 2, 3, 5, 7, or 10 degrees to about 15, 20, 25, 30, 35, 40 or 45 degrees.

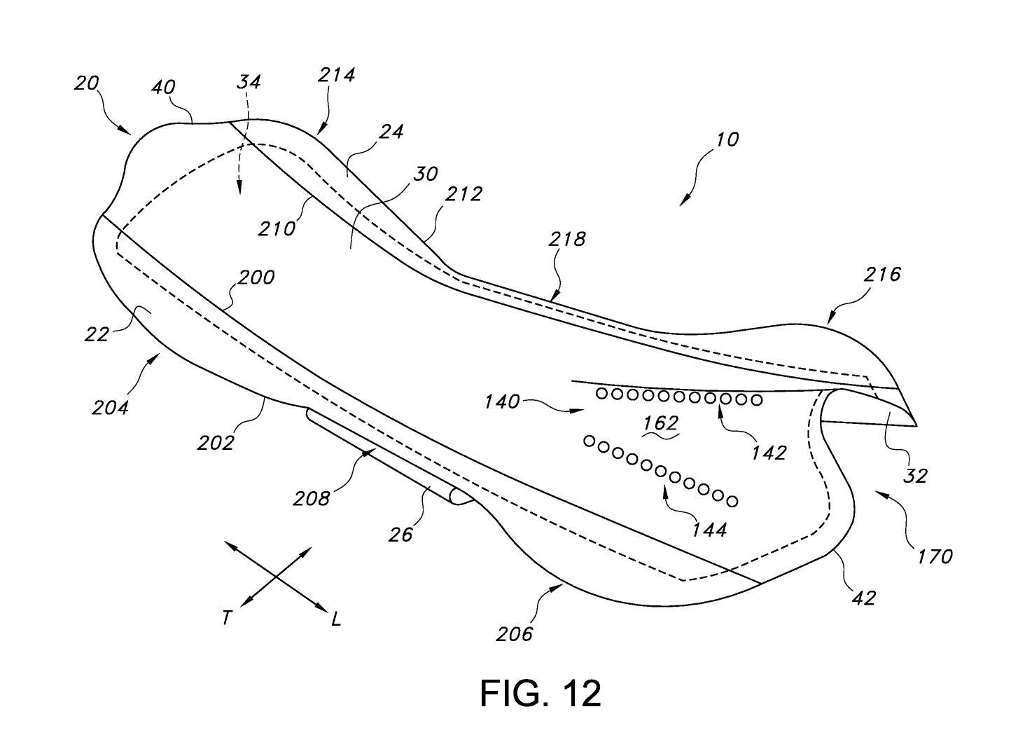

Referring to FIG. 12, the absorbent article 10 of FIG. 1 is illustrated in a tented configuration 170. The tented configuration 170 is formed in the posterior region of the absorbent article 10 when the absorbent article 10 is subjected to lateral compressive force. The tented configuration 170 is formed relative to the first flexure 142, the second flexure 144, and the third flexure 146 (not visible in this view). The tented configuration 170 is adapted to conform to the perineal region posterior to the vulva for the purpose of intercepting body exudates moving along the skin and/or preventing body exudates from following the body beyond the vulva region. The tented configuration 170 includes the topsheet layer 30, the backsheet layer 32, and the absorbent system 34 positioned between the topsheet layer 30 and the backsheet layer 32. The tented configuration 170 is shaped by the first flexure 142, the second flexure 144, and the third flexure 146. The tented configuration 170 can be dynamic due to a lack of a garment attachment 230 on the exterior surface 78 of the backsheet layer 32 of the absorbent article 10 in the area of the backsheet layer 32 below the flexure feature 140. The lack of the garment attachment 230 in such an area can result in a lack of attachment between the absorbent article 10 and the wearer's undergarment in such an area thereby allowing the absorbent article 10 to be dynamic in use and form a tented configuration 170. The first flexure 142 is believed to act as a lever to assist in lifting the tented configuration 170 to provide contact against the perineum and posterior vulva. The lifting force is generated by the reciprocating motion of the legs. This reciprocating motion alternately pumps the first side 162 of the dynamic region 160 and the second side 164 (not visible in this view) of the dynamic region 160 towards the gluteal cleft. Generally, the compressive force of the legs alternates when walking or running. In these situations, the compressive force alternates between the two sides of the absorbent article 10. The compression force can be transferred to the dynamic region 160 in the posterior region of the absorbent article 10. The first side 162 and the second side 164 are free to move in response to these forces because the backsheet layer 32 is free of a garment attachment 230 in the area of the backsheet layer 32 below the flexure feature 140.

### Side Covers:

The absorbent article 10 can have an opposing pair of non-integral side covers, 22 and 24. In various embodiments, such as, for example, illustrated in FIGS. 1-8 the non-integral side covers, 22 and 24, can be provided as two separate components which can be bonded to the absorbent article 10. Each side cover, 22 and 24, can extend in the longitudinal direction (L) and can extend the length of the chassis 20 of the absorbent article 10 from the first transverse direction end edge 40 to the second transverse direction end edge 42 of the chassis 20. In such embodiments, the side covers, 22 and 24, can be positioned over the longitudinal direction bond regions, 110 and 112, which form a portion of the seal of the chassis 20. The side covers, 22 and 24, can be bonded to a portion of the exterior surface 78 of the backsheet layer 32 and/or a portion of the wearer facing surface 98 of the topsheet layer 30. For example, referring to FIGS. 1-3, the side covers, 22 and 24, can be bonded to at least a portion of the exterior surface 78 of the backsheet layer 32. As an additional example, referring to FIGS. 4-6, the side covers, 22 and 24, can be bonded to at least a portion of the wearer facing surface 98 of the topsheet layer 30. Such side covers, 22 and 24, can be adhesively, thermally, ultrasonically, or otherwise bonded to the chassis 20. Traditional absorbent article construction adhesive may be used to bond the side covers, 22 and 24, to the chassis 20. In such a positioning, the side covers, 22 and 24, can provide a softer and less irritating material to be in contact with the skin of the wearer than the exterior surface 78 of the backsheet layer 32. In such a positioning, the side covers, 22 and 24, can prevent the longitudinal direction bond regions, 110 and 112, from coming into direct contact with the skin of the wearer. In such a positioning, when the wings, 26 and 28, of the absorbent article 10 are wrapped about a wearer's undergarment, the side covers, 22 and 24, which are independent from the wings, 26 and 28, can remain undistorted by the folding of the wings, 26 and 28, about the wearer's undergarment.

In various embodiments, such as embodiments in which the side covers, 22 and 24, are provided as two separate components bonded to the absorbent article 10, such as, for example, illustrated in FIGS. 1-8, each of the side covers, 22 and 24, can have a longitudinal direction edge, 200 and 210, respectively, which is positioned closer to the longitudinal axis 12 of the absorbent article 10 and can have a longitudinal direction distal edge, 202 and 212, respectively, which is positioned further from the longitudinal axis 12 of the absorbent article 10 than the longitudinal direction edge, 200 and 210, respectively. In various embodiments, while the longitudinal direction edges, 200 and 210, of the side covers, 22 and 24, respectively, can be bonded to the absorbent article 10, the distal edges, 202 and 212, of the side covers, 22 and 24, respectively, can remain unbonded to the absorbent article 10. In various embodiments, each of the longitudinal direction edges, 200 and 210, can be linear. In various embodiments, each of the longitudinal direction edges, 200 and 210, can have a curve (convex and/or concave). In various embodiments, each of the longitudinal direction distal edges, 202 and 212, can be linear. In various embodiments, each of the longitudinal direction distal edges, 202 and 212, can have a curve (convex and/or concave). In various embodiments, each of the longitudinal direction edges, 200 and 210, can be linear and each of the longitudinal direction distal edges, 202 and 212, can have a curve (convex and/or concave). In various embodiments, each of the longitudinal direction edges, 200 and 210, and each of the longitudinal direction distal edges, 202 and 212, can have a curve (convex and/or concave). In various embodiments, each of the longitudinal direction edges, 200 and 210, can have a curve (convex and/or concave) and each of the longitudinal direction distal edges, 202 and 212, can be linear. In various embodiments, each of the longitudinal direction edges, 200 and 210, and each of the longitudinal direction distal edges, 202 and 212, can be linear.

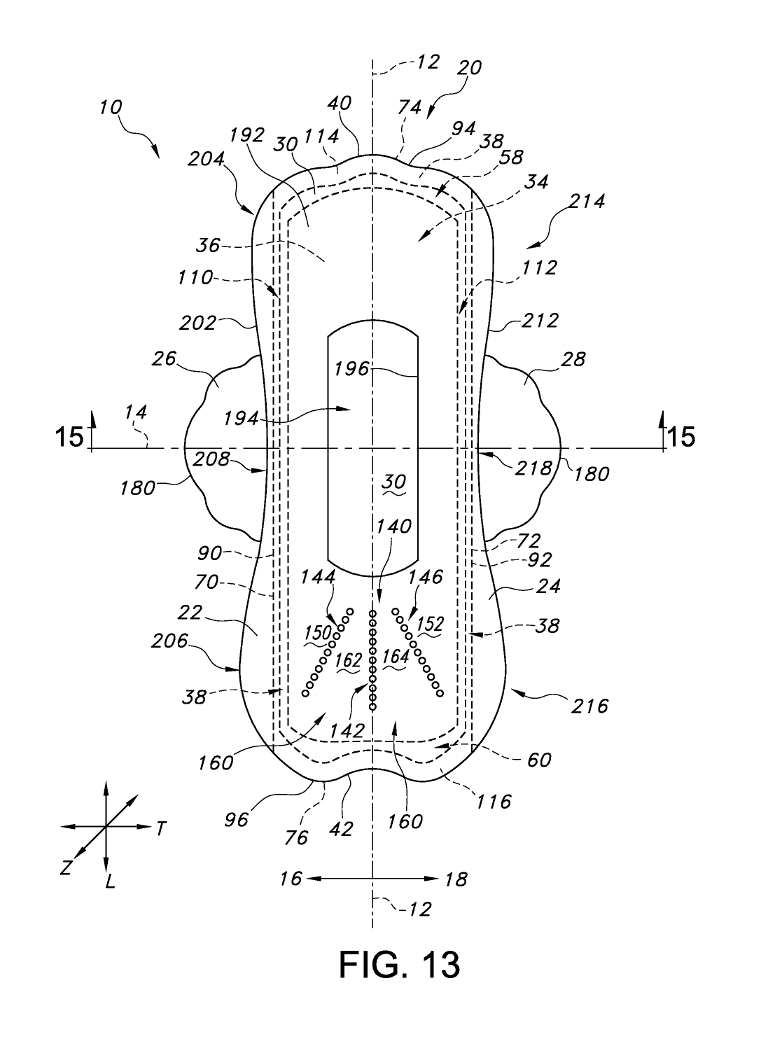

In various embodiments, such as, for example, illustrated in FIGS. 13-15 the non-integral side covers, 22 and 24, can be extensions of, and integral with, a cover bridge 192 which can be bonded to the absorbent article 10. Each side cover, 22 and 24, and the cover bridge 192 can extend in the longitudinal direction (L) and can extend the length of the chassis 20 of the absorbent article 10 from the first transverse direction end edge 40 to the second transverse direction end edge 42 of the chassis 20. In such embodiments, the side covers, 22 and 24, can be positioned over the longitudinal direction bond regions, 110 and 112, which form a portion of the seal of the chassis 20 and the cover bridge 192 can be positioned over at least the topsheet layer 30 of the chassis 20. The cover bridge 192 can be constructed of any material described herein as suitable for the topsheet layer 30. In various embodiments, the cover bridge 192 can be provided with an opening 194, defined by a perimeter 196, through which the topsheet layer 30 can be exposed. The opening 194 can be positioned at various positions along the longitudinal axis 12 and transverse axis 14 as deemed suitable for the intake of body exudates into the absorbent article 10. The opening 194 can have any shape as well as any length and width as deemed suitable for the intake of body exudates into the absorbent article 10.

In various embodiments, the cover bridge 192 and/or the extensions of the cover bridge 192 forming the side covers, 22 and 24, can be bonded to a portion of the exterior surface 78 of the backsheet layer 32 and/or a portion of the wearer facing surface 98 of the topsheet layer 30. For example, referring to FIGS. 13-15, the cover bridge 192 can be bonded to the topsheet layer 30 of the absorbent article 10 and the side covers, 22 and 24, as extensions of the cover bridge 192, can have a portion which remains unbonded to the absorbent article 10. It is to be understood that in various embodiments in which the chassis 20 of the absorbent article 10 is constructed such as illustrated, for example, in FIGS. 1-3, in which the backsheet layer 32 partially encloses the absorbent system 34, the cover bridge 192 can be bonded to a portion of the wearer facing surface 98 of the topsheet layer 30 and/or a portion of the exterior surface 78 of the backsheet layer 32 of the chassis 20 of the absorbent article 10. In such embodiments, the extensions of the cover bridge 192 forming the side covers, 22 and 24, can have a portion which can remain unbonded to the absorbent article 10. The cover bridge 192 and/or a portion of the side covers, 22 and 24, can be adhesively, thermally, ultrasonically, or otherwise bonded to the chassis 20. Traditional absorbent article construction adhesive may be used to bond the cover bridge 192 and/or a portion of the side covers, 22 and 24, to the chassis 20. In such a positioning, the side covers, 22 and 24, can provide a softer and less irritating material to be in contact with the skin of the wearer than the exterior surface 78 of the backsheet layer 32. In such a positioning, the side covers, 22 and 24, can prevent the longitudinal direction bond regions, 110 and 112, from coming into direct contact with the skin of the wearer. In such a positioning, when the wings, 26 and 28, of the absorbent article 10 are wrapped about a wearer's undergarment, the side covers, 22 and 24, which are independent from the wings, 26 and 28, can remain undistorted by the folding of the wings, 26 and 28, about the wearer's undergarment.

In various embodiments, such as embodiments in which the side covers, 22 and 24, are provided as extensions of a cover bridge 192, each of the side covers, 22 and 24, can have a longitudinal direction distal edge, 202 and 212, respectively, which is positioned further from the longitudinal axis 12 of the absorbent article 10. In various embodiments, each of the longitudinal direction distal edges, 202 and 212, can be linear. In various embodiments, each of the longitudinal direction distal edges, 202 and 212, can have a curve (convex and/or concave).

In various embodiments, whether provided as two separate components or as extensions of a cover bridge 192, the longitudinal direction distal edges, 202 and 212, of the side covers, 22 and 24, respectively, can each have at least one convex curve, such as convex curves, 204 and 214, respectively, and at least one concave curve, such as concave curves, 208 and 218, respectively. In various embodiments, the longitudinal direction distal edges, 202 and 212, of the side covers, 22 and 24, respectively, can each at least two convex curves. In such embodiments, longitudinal direction distal edge 202 can have convex curves, 204 and 206, and longitudinal direction distal edge 212 can have convex curves, 214 and 216. In such embodiments, convex curves, 204 and 206, can be separated by concave curve 208. In such embodiments, convex curves, 214 and 216, can be separated by concave curve 218. A curve can be defined as convex or concave when viewed from the perspective of the intersection of the longitudinal axis 12 and the transverse axis 14.

In various embodiments such as when the side covers, 22 and 24, are provided as two separate components, the transverse direction (T) width of the side covers, 22 and 24, as measured between the longitudinal direction edges, 200 and 210, respectively, and the maximum point of convex curves, 204 and 214, respectively, can be the same as or smaller than the transverse direction (T) width of the side covers, 22 and 24, as measured between the longitudinal direction edges, 200 and 210, respectively, and the maximum point of convex curves, 206 and 216, respectively. In various embodiments such as when the side covers, 22 and 24, are provided as extensions of cover bridge 192, the longitudinal direction distal edges, 202 and 212, in the area of convex curves, 204 and 214, respectively, can substantially align with the longitudinal direction distal edges, 202 and 212 in the area of convex curves, 206 and 216, respectively, or they can be closer to the longitudinal axis 12 of the absorbent article 10 than the longitudinal direction distal edges, 202 and 212, in the area of the convex curves, 206 and 216.

In various embodiments, the side covers, 22 and 24, can be constructed from a material which can be the same as or different from the material of the topsheet layer 30 of the chassis 20. In various embodiments, the topsheet layer 30 of the chassis 20 can be at least partially hydrophilic and the side covers, 22 and 24, may be inherently hydrophobic or may be treated with a hydrophobic coating. In various embodiments, the side covers, 22 and 24, can be constructed from a material which can be the same as or different from the material of the cover bridge 192, if present. In various embodiments, the cover bridge 192 can be at least partially hydrophilic and the extensions forming the side covers, 22 and 24, may be inherently hydrophobic or may be treated with a hydrophobic coating.

Different nonwoven, woven, or film sheet materials may be utilized as the side covers, 22 and 24. The selection of such side cover, 22 and 24, materials can vary based upon the overall desired attributes of the side covers, 22 and 24. For example, it may be desired to have a hydrophilic material in the topsheet layer 30 of the chassis 20 and hydrophobic-barrier type materials in the side covers, 22 and 24, to prevent leakage and increase a sense of dryness in the area of the side covers, 22 and 24. Either of the topsheet layer 30 of the chassis 20 and/or the side covers, 22 and 24, may be treated with surfactants and/or skin-health benefit agents, as are well known in the art.

Such longitudinally directed side covers, 22 and 24, can be of a single or multi-layered construction. In various embodiments, the side covers, 22 and 24, can be adhesively or otherwise bonded laminates. In various embodiments, the side covers, 22 and 24, can be a through air bonded carded web material. In various embodiments, the side covers, 22 and 24, can be a single layer of material, such as through air bonded carded web, without a second layer of material, such as a barrier film material, present. In various embodiments, the side covers, 22 and 24, can be constructed of an upper fibrous nonwoven layer, such as a spunbond material, laminated to a bottom layer of a hydrophobic barrier film material. Such a spunbond layer may be formed from a polyolefin, such as a polypropylene and can include a wetting agent if desired. In various embodiments, a spunbond layer can have a basis weight from about 10 or 12 gsm to about 30 or 70 gsm and can be treated with hydrophilic wetting agents. In various embodiments, a film layer may have apertures to allow fluid to permeate to lower layers, and may be either of a single layer or multi-layer construction. In various embodiments, such film can be a polyolefin, such as a polyethylene having a basis weight from about 10 to about 40 gsm. Construction adhesive can be utilized to laminate the spunbond layer to the film layer at an add-on level of between about 0.1 gsm and 15 gsm. When a film barrier layer is used in the overall side cover, 22 and 24, design, it may include opacifying agents, such as film pigments, that can help the film in masking stains along the absorbent article 10 side edges, thereby serving as a masking element. In such a fashion, the film layer can serve to limit visualization of a fluid insult stain along the absorbent article 10 side edges when viewed from above the absorbent article 10. In various embodiments, the side covers, 22 and 24, can be laminates such as a spunbond-meltblown-meltblown-spunbond layer (“SMMS”) laminate, spunbond-film laminate, or alternatively, other nonwoven laminate combinations.

### Wings:

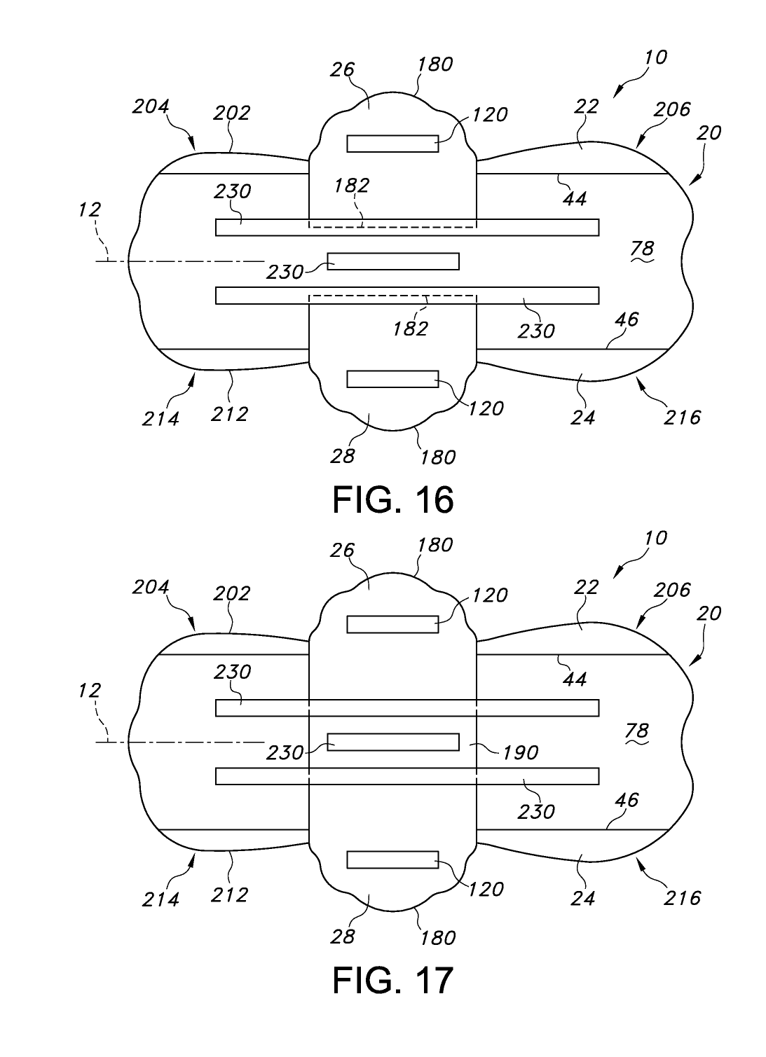

The absorbent article 10 can have a pair of non-integral wings, 26 and 28, extending outwardly in the transverse direction (T) from the absorbent article 10. The wings, 26 and 28, can be bonded to the exterior surface 78 of the backsheet layer 32 of the chassis 20 of the absorbent article 10. Referring to FIG. 16, in various embodiments, the wings, 26 and 28, can be two separate components bonded to the exterior surface 78 of the backsheet layer 32. In such an embodiment, each wing, 26 and 28, can have a distal edge 180 extending outward in a transverse direction (T) from the absorbent article 10 and proximal edge 182 positioned closer to and/or adjacent to the longitudinal axis 12 of the absorbent article 10 or between the longitudinal direction side edges, 44 and 46, and the longitudinal axis 12 of the absorbent article. The proximal edge 182 can be permanently bonded to the backsheet layer 32 of the absorbent article 10 while the distal edge 180 of the wings, 26 and 28, can remain unbonded to the backsheet layer 32 of the absorbent article 10. Referring to FIG. 17, in various embodiments, the wings, 26 and 28, can be extensions of, and integral with, a wing bridge 190 which can be bonded to the backsheet layer 32 and extend in a transverse direction across the width of the chassis 20 of the absorbent article 10. In such an embodiment, each wing can have a distal edge 180 extending outward in a transverse direction (T) from the absorbent article 10. The wing bridge 190 can be permanently bonded to the backsheet layer 32 of the absorbent article in the vicinity of the longitudinal axis 12 of the absorbent article 10 and the distal edges 180 of each of the wings, 26 and 28, can remain unbonded to the backsheet layer 32 of the absorbent article 10.

In various embodiments, whether the wings, 26 and 28, are provided as two separate components or as extensions from a wing bridge 190, the wings, 26 and 28, can be provided in any manner as deemed suitable so as to be adjustable when the absorbent article 10 is positioned for usage within a wearer's undergarment. For example, a permanent bonding of the wings, 26 and 28, to the absorbent article 10 can be located in the vicinity of the longitudinal axis 12 of the absorbent article 10. In various embodiments, the wings, 26 and 28, could be provided with temporary and releasable bonds with the backsheet layer 32 a distance away from the permanent bond in the immediate vicinity of the longitudinal axis 12. The temporary and releasable bonds can be broken by the wearer of the absorbent article 10 to customize and adjust the amount of wing coverage needed for placement of the absorbent article 10 in their undergarment. In various embodiments, the wings, 26 and 28, could be provided with pleats which can be releasably bonded together to remain in a folded configuration prior to usage of the absorbent article 10. The releasably folded configuration of the pleats can allow the wearer of the absorbent article 10 to extend, or refrain from extending, the wings, 26 and 28, as needed to conform to the width of the undergarment of the wearer.

The wings, 26 and 28, can be constructed from materials described above with respect to the topsheet layer 20 and the backsheet layer 22. The wings, 26 and 28, can be formed independently and separately attached to an intermediate section of the absorbent article 10. Wings, 26 and 28, that are made independent of the other components of the absorbent article 10 can be bonded to a portion of the backsheet layer 32. The wings, 26 and 28, can be bonded to the absorbent article 10 in locations which may not interfere with and/or hinder further manipulation of the absorbent article 10, such as, for example, the folding of the absorbent article 10 by the manufacturer to place the absorbent article 10 into the final packaged configuration. Examples of processes for manufacturing absorbent articles 10 and wings, 26 and 28, include, but are not limited to, those described in U.S. Pat. No. 4,059,114 to Richards, U.S. Pat. No. 4,862,574 to Hassim, et al., U.S. Pat. No. 5,342,647 to Heindel, et al., U.S. Pat. No. 7,070,672 to Alcantara, et al., U.S. Publication No., 2004/0040650 to Venturino, et al., and international publication WO1997/040804 to Emenaker, et al., each of which are hereby incorporated by reference thereto in its entirety. Each of the wings, 26 and 28, can have an attachment aid 120, such as, for example, a garment attachment adhesive or a hook, to keep the absorbent article 10 securely and properly positioned in the undergarment. The wings, 26 and 28, can wrap around the crotch region of the wearer's undergarment to aid in securing the absorbent article 10 to the wearer's undergarment when in use. Each wing, 26 and 28, can fold under the crotch region of the wearer's undergarment and the attachment aid 120 can either form a secure attachment to the opposite wing, 26 or 28, or directly to the surface of the wearer's undergarment.

### Garment Attachment:

The absorbent article 10 can be provided with a garment attachment 230 which can be located on an exterior surface 78 of the backsheet layer 32 for attachment of the absorbent article 10 to a wearer's undergarment. The garment attachment 230 can be provided in any suitable arrangement and/or pattern on the exterior surface of the backsheet layer 32 as deemed suitable. In various embodiments, such as embodiments in which the absorbent article 10 has a flexure feature 140, the backsheet layer 32 can be substantially free of a garment attachment 230 in the area of the backsheet layer 32 which is below the flexure feature 140. In such embodiments wherein the backsheet layer 32 has an area which is substantially free of a garment attachment 230, that area of the absorbent article 10 is substantially unattached to the wearer's undergarment and can move in response to the alternating movement of the legs thereby inducing the absorbent article 10 to re-position into a tented configuration 170. The garment attachment 230 can include any suitable attachment mechanism, such as, but not limited to, adhesive, cohesive, hooks, snaps, clips, or the like, or combinations thereof.

In various embodiments, the garment attachment 230 can be provided in any suitable amount on the exterior surface 78 of the backsheet layer 32 as deemed suitable. In various embodiments, the garment attachment 230 can be applied over the complete area of the exterior surface 78 of the backsheet layer 32. In various embodiments, the garment attachment 230 can be provided on less than the complete area of the exterior surface 78 of the backsheet layer 32. In various embodiments, the garment attachment 230 can be provided in any pattern as deemed suitable, such as, for example, stripes, swirls, dots, or the like, and combinations thereof.

In various embodiments, the garment attachment 230 can be provided in an amount that can correspond to the transverse direction (T) width of the crotch region of a wearer's undergarment. In various embodiments, an absorbent article 10 can be intended to be worn in an undergarment with a wide transverse direction (T) crotch region and the absorbent article 10 can be provided with a garment attachment 230 in an amount which can correspond to the transverse direction (T) width of the wide crotch region of the undergarment. In various embodiments, an absorbent article 10 can be intended to be worn in an undergarment with a narrow transverse direction (T) crotch region and the absorbent article 10 can be provided with a garment attachment 230 in an amount which can correspond to the transverse direction (T) narrow width of the crotch region of the undergarment. In such embodiments, the absorbent article 10 can have a wearer facing surface (topsheet layer 30 and side covers, 22 and 24) which can have a transverse direction (T) width which can be wider than the transverse direction (T) width of the absorbent system 34 which can be wider than the transverse direction (T) width of the garment attachment 230.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article characterized by comprising:
a. a longitudinal direction, a transverse direction, and a depth direction;
b. a longitudinal axis and a transverse axis wherein the absorbent article is asymmetrical about the transverse axis;
c. a chassis comprising:
i. a first end edge and a second end edge;
ii. a topsheet layer comprising a first longitudinal direction peripheral edge and a second longitudinal direction peripheral edge;
iii. a backsheet layer comprising a first longitudinal direction peripheral edge and a second longitudinal direction peripheral edge;
iv. an absorbent system positioned between the topsheet layer and the backsheet layer, the absorbent system comprising an absorbent core, a garment facing surface, a wearer facing surface, and a first longitudinal direction peripheral region and a second longitudinal direction peripheral region;
v. a first longitudinal direction bond region wherein the first longitudinal direction peripheral edge of the topsheet layer is bonded to the first longitudinal direction peripheral edge of the backsheet layer; and
vi. a second longitudinal direction bond region wherein the second longitudinal direction peripheral edge of the topsheet layer is bonded to the second longitudinal direction peripheral edge of the backsheet layer;
d. a first side cover bonded to the chassis in the region of the first longitudinal direction bond region and a second side cover bonded to the chassis in the region of the second longitudinal direction bond region; and
e. a first non-integral wing and a second non-integral wing, each of the first and second non-integral wings bonded to the backsheet layer.

2. The absorbent article of claim 1 wherein the first non-integral wing and the second non-integral wing are separate components from each other.

3. The absorbent article of claim 1 wherein the first non-integral wing and the second non-integral wing are integral with a bridge which is bonded to the backsheet layer.

4. The absorbent article of claim 1 wherein the first side cover and the second side cover are separate components from each other.

5. The absorbent article of claim 1 wherein the first side cover and the second side cover are integral with a bridge which is bonded to the topsheet layer.

6. The absorbent article of claim 1 wherein the first longitudinal direction bond region is located in the vicinity of the first longitudinal direction peripheral region and the second longitudinal direction bond region is located in the vicinity of the second longitudinal direction peripheral region.

7. The absorbent article of claim 1 wherein the first longitudinal direction bond region and the second longitudinal direction bond region each overlay a portion of the wearer facing surface of the absorbent system.

8. The absorbent article of claim 1 wherein each of the first side cover and the second side cover is bonded to an exterior surface of the backsheet layer.

9. The absorbent article of claim 8 wherein each of the first side cover and the second side cover is further bonded to a portion of the topsheet layer.

10. The absorbent article of claim 1 wherein each of the first side cover and the second side cover extend between the first end edge and the second end edge of the chassis.

11. The absorbent article of claim 1 wherein each of the first side cover and the second side cover have at least one concave region and at least convex region.

12. The absorbent article of claim 1 wherein the first end edge has at least one convex region and the second end edge has at least one concave region.

13. The absorbent article of claim 12 wherein the first end edge and the second end edge form a nesting configuration.

14. The absorbent article of claim 1 wherein the chassis further comprises at least one flexure feature.

15. The absorbent article of claim 14 wherein the at least one flexure feature comprises at least one flexure element extending in a direction generally parallel to the longitudinal axis.

16. The absorbent article of claim 15 wherein the at least one flexure feature comprises a second flexure element, the second flexure element spaced in the transverse direction outward from the first flexure element and positioned at an angle relative to the longitudinal axis.

17. The absorbent article of claim 14 further comprising a garment attachment on an exterior surface of the backsheet layer wherein the backsheet layer is substantially free of the garment attachment in an area of the backsheet layer positioned below the flexure feature in the depth direction of the absorbent article.

18. The absorbent article of claim 1 wherein the absorbent core is a single layer of material.

19. The absorbent article of claim 1 wherein the absorbent core is formed of two layers of material.

20. The absorbent article of claim 1 wherein the absorbent system further comprises at least one additional layer selected from a surge layer, a fluid intake layer, a transfer delay layer and a distribution layer.
